(19) United States
(12) Patent Application Publication
(10) Pub. No.: US 2017/0275680 A1
(43) Pub. Date: Sep. 28, 2017

(54) Methods and Apparatus for Nanoparticle-assisted Nucleic Acid Amplification, Hybridization and Microarray Analysis

(71) Applicants: Paul Chi Hang Li, Coquitlam (CA); Abootaleb Sedighi, Shiraz (IR)

(72) Inventors: Paul Chi Hang Li, Coquitlam (CA); Abootaleb Sedighi, Shiraz (IR)

(21) Appl. No.: 15/080,531

(22) Filed: Mar. 24, 2016

(51) Int. Cl. C12Q 1/68

(52) U.S. Cl. CPC C12Q 1/6837; B82Y 15/00

(57) ABSTRACT

Nucleic acid hybridization methods are disclosed. An example method comprises: immobilizing probe nucleic acid molecules on a surface; flowing target nucleic acid molecules to the immobilized probe nucleic acid molecules on said surface in a hybridization buffer solution; washing said surface with a wash solution which comprises nanoparticles; and detecting the presence of duplexes on said surface comprising a strand of one of said target nucleic acid molecules and a strand of one of said probe nucleic acid molecules. In some embodiments, the target nucleic acid molecules are generated using a helicase-dependent amplification method wherein the reaction solution comprises nanoparticles.

### CROSS-REFERENCE TO RELATED APPLICATION

This application claims priority to U.S. Provisional Application No. 62/144,827, filed Apr. 8, 2015. The content of the priority application is incorporated by reference in its entirety.

### TECHNICAL FIELD

The present invention relates to methods and apparatus for nucleic acid amplification, hybridization and microarray analysis.

### BACKGROUND

Nucleic acid diagnostics is currently the fastest growing segment of the in vitro diagnostics market. However, with the perspective of personalized medicine in the future, these diagnostic techniques must be simple, fast, and especially, reliable. DNA hybridization is a promising tool for nucleic acid diagnostics because the method is simple and has a high sample-throughput potential. However, hybridization assays are limited by an inherently low specificity, which is the main cause of discrepancies in the assay results. This limitation is aggravated for single nucleotide polymorphism (SNP) analysis in which the mismatched target strand (MM) varies from the perfectly matched target (PM) strand by only a single base pair. In order to improve the assay specificity, DNA hybridization, or subsequent wash step, is conventionally conducted at stringent conditions, when high temperature, low ionic strength or chemically denaturing medium is applied to reduce the nonspecific signal. These stringent conditions bring the duplexes near their melting temperatures, where a marginal difference in the duplex stability (i.e. PM vs. MM) causes a significant variation in their affinities. However, this high-temperature method is not effective when conducted for highly multiplexed analyses, such as DNA microarrays where many thousands of targets, each with its own melting temperature, have to be analysed simultaneously at a single optimized temperature. Therefore, low specificity with false-positive and false-negative outcomes is resulted for those targets with melting temperatures far from the hybridization temperature. These faults are widely agreed to be the main pitfall to impact the accuracy of the DNA microarray platform, resulting in a barrier for its adoption for clinical applications.

Various novel methods to boost specificity are reported in which special hybridization probes are designed to work at temperatures well below the melting temperatures. For instance, when oligonucleotide probes with short lengths are used, the nonspecific binding is thermodynamically less favorable, leading to an improved sensitivity while boosting specificity. However, the design of these special probes is usually complicated and the method of using them is not compatible with multiplex analyses.

Additionally, nucleic acid amplification is an integral part of molecular diagnostics. The polymerase chain reaction (PCR), invented in 1980s, made a significant contribution in the area of molecular biology and molecular diagnostics. PCR is a powerful technique and is still considered the gold standard for nucleic acid amplification. However, the need for thermocycling in PCR limits its use in certain settings (e.g. in limited-resources or point-of-care environments). An alternative to thermocycling is the isothermal method, which includes a variety of techniques such as loop-mediated amplification (LAMP), rolling-circle amplification (RCA), nucleic acid sequence-based amplification (NASBA), strand displacement amplification (SDA), and helicase-dependent amplification (HDA). HDA, which uses helicase instead of heat to denature double-stranded DNA, is considered a true isothermal technique because the entire process occurs at a single temperature. However, the rate-limiting step of HDA is denaturation, and so the method is limited by the low denaturation efficiency of the helicase. This limitation is supported in the literature, showing that HDA has been successfully used to amplify more for the shorter bacterial DNA and viral cDNA, and less for the longer DNA, such as human DNA.

Based on the foregoing, it would be desirable to provide improved nucleic acid hybridization and microarray methods and apparatus. For example, it would be desirable to provide nucleic acid hybridization and microarray methods and apparatus with enhanced specificity of nucleic acid hybridization without reducing detection sensitivity.

It would also be desirable to provide improved isothermal methods for nucleic acid amplification. For example, it would be desirable to provide HDA (helicase-dependent amplification) methods with improved efficiency and sensitivity.

### BRIEF SUMMARY OF THE INVENTION

One aspect of the invention provides a method for nanoparticle-assisted nucleic acid hybridization analysis. An example embodiment of the nucleic acid hybridization method comprises a number of steps. In a first step, probe nucleic acid molecules are immobilized on a surface. The surface may be a solid surface, or a semi-solid surface. For example, the surface may be a gel, polyacrylamide, agar, agarose, or gelatin. The surface may be made of solid or curable materials, for example, glass, silicon, plastic, polymer, cellulose, etc. The solid surface may be, for example, a solid surface inside a test tube or a microfluidic channel, or on a glass slide, a test chip, a microarray chip, a microtiter plate, a nylon membrane, or a film. The surface may be substantially flat, or curved, for example, the surface of a well of a microtiter plate, or the surface (hereafter refer as crust) of a spherical bead. The surface may also be coated or conjugated with one or more compounds, for example, the surface may be aldehyde-functionalized.

There are a number of ways to immobilize or tether probe nucleic acid molecules on the surface. One approach is in situ synthesis, wherein probe nucleic acid molecules are synthesized directly base by base on the surface. Another approach is to spot or print the probe nucleic acid molecules on the surface using contact or non-contact printing methods. Other methods of immobilizing probe nucleic acid molecules are known to persons skilled in the art. For example, immobilization can be achieved by chemical, mechanical, or biochemical methods such as covalent binding, adsorption, polymer encapsulation and so forth. One example method of chemical immobilization is Schiff-base linkage formed between an aminated DNA or oligonucleotides probe and an aldehyde-functionalized glass surface.

The probe nucleic acid molecules are typically single-stranded, or comprise at least a single-stranded region. In some embodiments, the probe nucleic acid molecules may also comprise a double-stranded region, or a triple-stranded region. The probe nucleic acid molecules may be formed from oligonucleotides, deoxyribonucleic acid (DNA), ribonucleic acid (RNA), or peptide nucleic acid (PNA). They may include both natural or artificial or synthetic nucleic acids. They may include genomic DNA or even a chromosome preparation (e.g., a chromosome preparation suitable for fluorescent in situ hybridization (FISH)). They may be synthesized or generated or amplified using standard procedures known to those skilled in the art or ordered from commercial vendors. Standard molecular biology methods for probe preparation can be found in Sambrook and Russel, Molecular Cloning: A Laboratory Manual, Cold Spring Harbor Laboratory Press, 2001, 3^{rd} edition, which is hereby incorporated by reference. In some embodiments, the probe nucleic acid molecules are between 10 and 1000 nucleotides in length. In some embodiments, the probe nucleic acid molecules are between 10 and 100 nucleotides in length. In some embodiments, the probe nucleic acid molecules are between 10 and 50 nucleotides in length.

After probe immobilization, in a second step, target nucleic acid molecules are flowed to the immobilized probe nucleic acid molecules on said surface in a hybridization buffer solution. The target nucleic acid molecules may be provided in the hybridization buffer solution, and the solution may be allowed to incubate on said surface for a period of time. This incubation will allow the hybridization of the target nucleic acid molecules with the immobilized probe nucleic acid molecules.

The target nucleic acid molecules may be oligonucleotides, deoxyribonucleic acid (DNA), ribonucleic acid (RNA) or an artificial or synthetic nucleic acid. They may be synthesized or generated or amplified using standard procedures known to those skilled in the art or ordered from commercial vendors. The target nucleic acid molecules may be isolated directly from samples (e.g. cells, tissues, cell extracts, tissue culture media, bodily fluids, environmental samples, other biological samples etc.), or they may first be amplified by polymerase chain reaction (PCR) or reverse-transcription PCR (RT-PCR) or an isothermal amplification method. In some embodiments, the target nucleic acid molecules may be amplicons amplified from a nucleic acid source (e.g., genomic DNA) using polymerase chain reaction (PCR) or an isothermal amplification method. An example isothermal amplification method may be a nanoparticle-assisted isothermal amplification method, which will be described in this disclosure. The target nucleic acid molecules may comprise synthetic, natural, or structurally modified nucleoside bases. The target nucleic acid molecule can also be from any source organism (e.g., human or another animal, virus, bacteria, insect, plant, etc.).

In some embodiments, the target nucleic acid molecules are between 10 to 1000 nucleotides or base-pairs in length. In some embodiments, the target nucleic acid molecules may exceed 1000 nucleotides or base-pair in length. The target nucleic acid molecules may comprise single-stranded molecules, or double stranded molecules, or combinations thereof. In some embodiments, the target nucleic acid molecules may comprise a single-stranded region and a double-stranded region. The target nucleic acid molecules may be purified or isolated molecules, or may be present in a solution or sample that comprises other molecules or contaminants. The target nucleic acid molecules may comprise nucleic acid molecules having different sequences (e.g., a mixture of genomic DNA molecules, a mixture of different PCR products, a mixture of cDNA molecules, or a mixture comprising two related DNA sequences differing by a single base-pair). The target nucleic acid molecules may be from a single sample source or may be from two or more sample sources (e.g., pooled cDNA molecules from two types of cells, one being stem cell, the other being differentiated cell, or genomic DNA from two human individuals).

The target nucleic acid molecules can either be unlabeled or they can be conjugated or otherwise coupled to a detectable label. Suitable detectable labels include, without limitation, fluorescent labels, redox (electrochemical) labels, and radioactive labels.

Coupling of a fluorescent label to nucleic acid molecules can be achieved using known nucleic acid-binding chemistry or by physical means, such as through ionic, covalent or other forces well-known in the art (see, e.g., Dattagupta et al., Anal. Biochem. 177:85-89 (1989): Saiki et al. Proc. Natl. Acad. Sci. USA 86:6230-6234 (1989); Gravitt et al. J. Clin. Micro. 36:3020-3027 (1998), each of which is hereby incorporated by reference in its entirety). Either a terminal base or another base near the terminal base can be bound to the fluorescent label. For example, a terminal nucleotide base of the target nucleic acid molecules can be modified to contain a reactive group, such as (without limitation) carboxyl, amino, hydroxyl, thiol, or the like.

The fluorescent label can be any fluorophore that can be conjugated to a nucleic acid and preferably has a photoluminescent property that can be detected and easily identified with appropriate detection equipment. Exemplary fluorescent labels include, without limitation, fluorescent dyes, semiconductor quantum dots, lanthanide element-containing complexes, and fluorescent proteins. Example fluorescent dyes include, without limitation, Calcein, FITC, Alexa™, Rhodamine 110, 5-FAM, Oregon Green™ 500, Oregon Green™ 488, RiboGreen™, Rhodamine Green™ Rhodamine 123, Magnesium Green™, Calcium Green™, Cy3™, Alexa™ 546, TRITC, Magnesium Orange™, Phycoerythrin R&B, Rhodamine Phalloidin, Calcium Orange™, Pyronin Y, Rhodamine B, TAMRA, Rhodamine Red™, ROX, Nile Red, YO-PRO™-3. R-phycocyanin, C-Phycocyanin, Cy5™, Thiadicarbocyanine, and Cy5.5™. Other dyes now known or hereafter developed may similarly be used.

Buffer conditions for hybridization are well-known to those skilled in the art and can be varied within relatively wide limits.

After hybridization of target nucleic acid molecules with probe nucleic acid molecules, in a third step, said surface is washed with a wash solution which comprises nanoparticles. The nanoparticles should be in suspended, non-aggregated form, or de-aggregated under suitable conditions. The nanoparticles may be sized between 1 and 100 nanometers. They may be spherical or rod-shaped or of other shapes. The nanoparticles may be coated with negatively charged ions. The negatively charged ions may help prevent aggregation of nanoparticles. The nanoparticles may be formed of a metal, a semiconductor, or an uncharged substrate, such as glass, or combinations thereof. The nanoparticles may be sized between 1 and 50 nm, or between 20 and 30 nm, or between 10 to 20 nm, or between 1 to 10 nm, or between 3.5 to 6.5 nm. The nanoparticles may have a mean particle size of 5.0 nm. The nanoparticles may have a coefficient of variance of particle size that is less than 15% of the mean particle size.

In some embodiments, the crusts of the nanoparticles are loaded with oligonucleotide stabilizers whose sequences are unrelated to the sequences of the probe nucleic acid molecules or the target nucleic acid molecules. The length of the oligonucleotide stabilizers may be 20-mer or shorter, or 15-mer or shorter, or 12-mer.

The concentration of the nanoparticles in the wash solution is in a range of 2 to 20 nM. In some embodiments, the concentration of NaCl in the wash solution may be in a range of 50 to 300 nM. In some embodiments, the wash solution has an ionic strength equivalent to NaCl concentration of between 50 and 150 nM.

In some embodiments, the washing step is performed at an ambient temperature. In some embodiments, the washing step is performed at a temperature below 30° C. In some embodiments, the washing step is performed at a temperature between 20° C. and 25° C.

The metal nanoparticles may be formed of a conductive metal or metal alloy that allows a nanoparticle to be capable of non-covalently associating with a single-stranded nucleic acid molecule. It should be appreciated that the colloidal suspension should maintain the metal nanoparticles in a stable environment in which they are substantially free of aggregation. The metal nanoparticles should not significantly associate with double-stranded nucleic acid molecules. Example metal nanoparticles include, without limitation, gold nanoparticles (AuNPs), silver nanoparticles, platinum nanoparticles, mixed metal nanoparticles (e.g., gold shell surrounding a silver core), and combinations thereof. The metal nanoparticles may be magnetic or magnetically attractable, for example, formed of an inner core such as cobalt and an outer layer such as gold.

Preparation of colloidal metal nanoparticle suspensions can be carried out according to known procedures, e.g., Grabar et al. Anal. Chem. 67:735-743 (1995), which is hereby incorporated by reference in its entirety. Metal nanoparticles may be stabilized in the solution by negatively charged anions, such as citrate, acetate, carbonate, phosphate, oxalate, sulfate, or nitrate.

In some embodiments, the nanoparticles comprise gold nanoparticles. Preparation of gold nanoparticles can be carried out according to known procedures, e.g., J. Turkevich, P. C. Stevenson, J. Hillier, Discuss. Faraday. Soc. 1951, 11, 55-75: J. Kimling, M. Maier, B. Okenve, V. Kotaidis, H. Ballot. A. Plech. J. Phys. Chem. B 2006, 110, 15700-15707; G. Frens, Colloid & Polymer Science, 1972, 250, 736-741; G. Frens, Nature (London). Phys. Sci. 1973, 241, 20-22; J. W. Slot and H. J. Geuze, Eur. J. Cell Biol. 38, 87 (1985); M. C. Daniels and D. Astruc. Chem. Rev. (Washington D.C.) 104, 293 (2004), each of which is hereby incorporated by reference in its entirety. Briefly, gold nanoparticles are typically produced in a liquid by reduction of chloroauric acid (HAuCl₄). After dissolving HAuCl₄, the solution is rapidly stirred while a reducing agent is added. This causes Au(III) ions to be reduced to neutral gold atoms. As more and more of these gold atoms form, the solution becomes supersaturated, and gold gradually starts to grow in the form of sub-nanometer particles. The rest of the gold atoms that form stick to the existing particles, and, if the solution is stirred vigorously enough, the particles will be fairly uniform in size. The anions in gold nanoparticle preparation also prevent the gold nanoparticles from aggregating. These anions may include citrate, acetate, carbonate, phosphate, oxalate, sulfate, or nitrate. Alternatively, the nanoparticles can be purchased from commercial sources. For example, gold nanoparticles can be purchased from Sigma Life Sciences.

Although some of the examples in the present disclosure describe experiments performed using gold nanoparticles, it will be appreciated by those skilled in the art that other nanoparticles having similar properties may also be used. For example, silver nanoparticles have been shown to behave like gold nanoparticles in terms of non-covalent binding with single-stranded nucleic acid molecules: Chen et al., Analyst, (2010), 135, 1066-1069, which is hereby incorporated by reference.

After washing, in a fourth step, a determination is made as to whether at least some of the target nucleic acid molecules have hybridized with probe nucleic acid molecules to form a hybridization duplex comprising a strand from the target nucleic acid molecules and a strand from the probe nucleic acid molecules and the level of hybridization. In some embodiments, the surface is dried before a detection method is applied. In some embodiments, a detection method may be applied without drying the surface. This determination or detection may be qualitative or quantitative. A large number of methods are available to detect or quantify hybridization duplexes on the surface. For example, if the target nucleic acid molecules were fluorescently labeled, the surface can be scanned for fluorescence emissions. For example, a confocal laser fluorescent scanner may be used. If both the target and the probe are fluorescently labeled, a detection method called fluorescence resonance energy transfer (FRET) may be used. Alternatively, if the target nucleic acid molecules comprise redox labels, or radioactive labels, other methods may be used to detect the level of hybridization. These methods are well known to those skilled in the art.

One aspect of the invention provides a method for distinguishing two target nucleic acid molecules whose nucleotide sequences differ by at least one nucleotide, the method comprising: carrying out two separate nucleic acid hybridization assays in parallel, the first assay with a first target and a probe, the second assay with a second target and the same probe, each assay comprising: providing a sample solution comprising a target nucleic acid; incubating said sample solution with probe nucleic acid molecules which are immobilized on a surface; washing said surface with a wash solution which comprises nanoparticles; and detecting the presence of target:probe duplex on the surface; whereby the two target nucleic acid molecules are distinguished by different degrees of hybridization to the probe.

The nanoparticle-assisted hybridization method can also be applied to microarray technology. A microarray is a multiplex technology commonly used in molecular biology. Procedures for microarrays are well-known to those skilled in the art: e.g., David Bowtell and Joseph Sambrook, DNA Microarrays: A Molecular Cloning Manual, Cold Spring Harbor Laboratory Press; 1st edition (2002), which is hereby incorporated by reference. A microarray consists of an arrayed series of tens, hundreds, thousands, or even tens of thousands of microscopic spots of picomoles (10⁻² moles) of oligonucleotides or DNA probes, each having a specific nucleotide sequence. These can be a short section of a gene or other DNA element that are used to hybridize a sample (e.g. cDNA or genomic DNA or RNA). Probe-target hybridization is usually detected and quantified by detection of fluorophore-, silver-, or chemiluminescence-labeled targets to determine relative abundance of nucleic acid sequences in the sample. Since an array can contain tens, hundreds, thousands, or even tens of thousands of probes, a microarray experiment can accomplish many tests in parallel.

One aspect of the invention provides a microarray method, the method comprising: providing a solid support; immobilizing a plurality of nucleic acid probes at discrete positions on the support; exposing a sample solution to the probes, the sample solution comprising sample nucleic acid molecules; washing off the sample solution with a wash solution which comprises nanoparticles; and determining the degree of hybridization between the sample molecules and the probes.

One aspect of the invention provides nanoparticle-assisted hybridization methods in association with a microfluidic microarray assembly (MMA) or a microchannel plate assembly. MMA and microchannel plate assemblies are described in WO 2006/060922 and L. Wang and P. C. H. Li, J. Agric. Food. Chem. 55, 10509 (2007), which are hereby incorporated by reference in their entirety. It should be noted that both MMA and microchannel plate assembly can be considered to be a subset of microarrays, and that microchannel plate assembly can be considered to be a subset of MMA.

In an embodiment, a method of using a microfluidic microarray assembly (MMA) comprises: providing a test chip; providing a first channel plate sealingly connectable to said test chip for applying at least one probe reagent to said test chip, wherein said first channel plate comprises a plurality of first microfluidic channels configured in a first predetermined reagent pattern; assembling said first channel plate to said test chip; flowing said at least one probe reagent through said first microfluidic channels to form a first array of said at least one probe reagent on said test chip in said first predetermined reagent pattern; immobilizing said at least one probe reagent on said test chip; removing said first channel plate from said test chip; providing a second channel plate sealingly connectable to said test chip for applying at least one sample reagent to said test chip, wherein said second channel plate comprises a plurality of second microfluidic channels configured in a second predetermined pattern differing from said first predetermined pattern; assembling said second channel plate to said test chip; flowing said at least one sample reagent through said second microfluidic channels to form a second array, wherein said second array intersects said first array at said test locations; flowing a wash solution which comprises nanoparticles through said second microfluidic channels; and detecting any hybridization products at said test locations.

In some embodiments, a plurality of different probes is used, and each of those probes is flowed through separate ones of the first microfluidic channels. In some embodiments, a plurality of different test samples are used, and each of those test samples is flowed through separate ones of the second microfluidic channels. In some embodiments, the first predetermined reagent pattern is a radial pattern and the second predetermined reagent pattern is a spiral pattern. In some embodiments, the first predetermined reagent pattern is a spiral pattern and the second predetermined reagent pattern is a radial pattern.

One aspect of the invention provides nanoparticle-assisted nucleic acid amplification methods. The methods may be isothermal amplification methods, wherein the nucleic acid is amplified at an isothermic temperature that does not require a thermal cycler. The isothermal amplification methods may be nanoparticle-assisted helicase-dependent isothermal nucleic acid amplification methods.

In an example embodiment, the nanoparticle-assisted helicase-dependent nucleic acid amplification method comprises these steps. First, double stranded substrate nucleic acid molecules are denatured by a helicase in a reaction solution which comprises nanoparticles. Then, primers are annealed to the denatured substrate nucleic acid molecules and are extended by a suitable polymerase to produce double-stranded nucleic acid molecules. The newly synthesized double-stranded nucleic acid molecules are then used as substrates by the helicase, entering the next round of the reaction. Thus, a chain reaction develops, resulting in exponential amplification of the substrate nucleic acid molecules. In some embodiments, the substrate nucleic acid molecules are digested with a suitable restriction enzyme to reduce the fragment size of the substrate nucleic acid molecules prior to denaturation by the helicase. For example, the substrate nucleic acid molecules may be digested by a restriction enzyme to a fragment size of less than 500 bp.

In conventional HDA (helicase-dependent amplification), helicase denatures dsDNA before DNA extension, and the rate of this method is limited by helicase's low denaturation efficiency. In this disclosure, we describe nanoparticle-assisted HDA (nanoHDA) which enhances the denaturation efficiency of conventional HDA by using nanoparticles (e.g., AuNPs). Nanoparticles with preferential affinity to ssDNA are utilised to improve helicase-mediated DNA denaturation. The same affinity of nanoparticles can also explain our observation that nanoparticles enhanced specificity by suppressing the formation of primer-dimers.

One aspect of the invention provides a combined method which couples a nanoparticle-assisted helicase-dependent isothermal nucleic acid amplification method with a nanoparticle-assisted nucleic acid hybridization method. The combined method comprises: amplifying a substrate nucleic acid in a helicase-dependent amplification (HDA) reaction in a reaction solution which comprises nanoparticles; purifying the amplified nucleic acid molecules; and using the amplified nucleic acid molecules as target molecules in a nanoparticle-assisted nucleic acid hybridization reaction (which is described in this disclosure). The concentration and other parameters of the nanoparticles used in the nanoHDA reaction and the nanoparticle-assisted nucleic acid hybridization reaction may be different and can be independently optimized.

### BRIEF DESCRIPTION OF DRAWINGS

In drawings which show non-limiting embodiments of the invention:

FIG. 1: Schematic diagram of the AuNP wash method used in a CD-NBA (CD-like NanoBioArray) chip, with one of the many spiral channels shown. The inset shows the destabilization enhanced by AuNPs at mismatched (MM), but not perfectly matched (PM), hybridization patches. The chip diagram is not drawn to scale.

FIG. 2: A) Fluorescence image of a part the CD-NBA chip showing the hybridization patches obtained from 12 spiral channels. These patches (200×100 μm) were resulted from the hybridization of 1 μL of A20 targets (10 nM) in the spiral channels with their corresponding perfectly matched (PM) and mismatched (MM) probes (A and W, respectively) pre-printed in a radial fashion on the chip. The hybridization step was performed at 22° C. with a spin rate of 900 rpm. The hybridization patches were either not washed (“no wash”), washed with 2 μL of the hybridization buffer (“stringent wash”) or washed with the hybridization buffer containing AuNPs of different sizes (5, 10, 12, 20 nm diameter) (“AuNP Wash”). The wash buffer were flowed in the spiral channel using a spin rate of 900 rpm (See FIG. 8 for an investigation on the effect of flow-mediated dynamic wash). Oligonucleotides of irrelevant sequences (10 nM, 20-mer) were loaded on the crusts of nanoparticles to stabilize them against salt-induced aggregation (B) The histogram shows the signal intensities of the hybridization patches obtained along the spiral target channels, with the specific signals (on PM probe lines) represented by the gray bar and nonspecific signals represented by white bars. The error bars show the standard deviations of 8 measurements. The line shows the specificity, which is determined by dividing the intensity of the PM patches by that of the MM patches (See FIG. 1). For DNA sequences of probes/targets and oligonucleotides stabilizers, see Table 2.

FIG. 3: Comparison of the stringent wash and AuNP wash methods in terms of sensitivity and specificity. A) The histogram shows the hybridization signals obtained after stringent wash. After DNA hybridization of A20 targets with their PM probes (A) and MM probes (W), the hybridization patches were washed with 2 μL of SSC buffer with concentrations from 0.01× to 2× (i.e. NaCl concentrations from 1.5 to 300 mM, respectively) at 3 different temperatures of 22, 30 and 40° C. For details, see FIG. 9. B) Histogram shows the hybridization signals obtained after the AuNP wash. The SSC 1× buffer solution (consisting of 150 mM of NaCl) contained 5-nm AuNPs with various concentrations of 0.2-40 nM. Error bars show the standard deviations of 10 measurements. For other conditions, see FIG. 2 C) The plot shows the correlation between signal-to-noise ratio (SNR) of the perfectly matched (PM) spots with their specificities (σ) for stringent wash and AuNP wash. The data were obtained from measurements using 4 different CD-NBA chips. The SNR values are the ratios of PM signal intensities over the average noise (˜480 fluorescence units). A SNR of 10 and a σ value of 2 was chosen as the minimum acceptable values. The plot area was divided into 4 regions showing low σ/low SNR (region 1), low σ/high SNR (region 2), high σ/low SNR (region 3) and high σ/high SNR (region 4).

FIG. 4: Optimization of salt content used in the AuNP wash method. Histograms of hybridization signals resulted from the washing of the hybridization patches using wash buffers containing different concentrations of NaCl (10-150 mM) at 22° C. The buffer solutions either contain (A) no AuNPs or (B) AuNP (5 nm) with a concentration of 5 nM. For other conditions see FIG. 2. C) Kinetics of the adsorption of Cy5-labeled 20-mer oligonucleotides (C-W20) onto 5-nm AuNPs in sodium citrate buffer (15 mM) at different NaCl concentrations from 0 to 150 mM. Each curve represents the normalized fluorescence by expressing the time-dependent fluorescence intensity as a fraction of the initial intensity. The rate of adsorption k′_{d} at each NaCl concentration, as obtained from the exponential fit of the normalized data, is shown beside the legend of the corresponding curve.

FIG. 5: Optimization of various experimental factors (e.g. oligonucleotide stabilizer and purine content of DNA targets) of the AuNP wash method. A) Histogram of the hybridization signals obtained after washing by SSC buffer solution (with 90 mM NaCl) containing AuNPs stabilized with different oligonucleotides. The 5-nm AuNPs (5 nM) were first stabilized with 12-mer and 20-mer oligonucleotides of different concentrations (8-20 nM for 12-mer and 5-20 nM for 20-mer). B) Histogram resulted from fluorescence intensity obtained at the hybridization patches of various targets following AuNP wash (12-mer stabilizer (8 nM), spin rate of 900 rpm). For other conditions see FIG. 5A and FIG. 2.

FIG. 6: Hybridization of PCR amplicons, with their corresponding perfectly matched (PM) and mismatched (MM) probes in the CD-NBA chip. A) shows the scanned fluorescence image and (B) shows the resulted histogram. The target molecules (80 base-pairs) were amplified from 4 different alleles of KRAS gene codon 12 and hybridized with their complementary probes preprinted on the chip surface. Each probe is perfectly matched with one of the targets and single base-pair mismatch with the other 3 targets. After hybridization, washing was conducted with a flow of wash buffer (SSC buffer with 90 mM NaCl) containing 5-nm AuNPs (5 nM, stabilized with 8 nM of 12-mer oligonucleotides) at a temperature of 22° C. and a spin rate of 900 rpm.

FIG. 7: The fluorescence image (A) and the resulted histogram (B) from DNA hybridization between PCR products (80 bp) amplified from 4 different genomic samples each contains DNAs with one of the alleles of KRAS gene codon 12 and the oligonucleotide probes immobilized on the surface of CD-NBA chip. The DNA targets were either free in the solution (free targets) or conjugated to the crust of AuNPs (AuNP targets). AuNP targets were prepared by mixing the PCR amplicons with 5-nm AuNPs and incubating the mix for 5 min. at 95° C. DNA hybridization experiments were performed by flowing of 1 μL of target solution (using a spin rate of 900 rpm and a temperature of 22° C.) in the spiral channels of CD-NBA chip. For DNA sequences of probes/targets, see Table 2.

FIG. 8: The fluorescence image of the hybridization signals (A), and the resulted histogram (B), following AuNP wash at different spin rates. The stop-flow wash was performed by incubation of the wash buffer (2 μL) within the spiral channels of CD-NBA chip for 15 min. The dynamic wash was performed by injection of 2 μL into the channels reservoirs and by flowing them in the channels at different spin rates (700-1500 rpm). In the CD-NBA chip, the liquid flow is driven by the centrifugal force, and the flow transports the AuNPs within the spiral channels of the chip and delivers the nanoparticles to the hybridization regions along the channel. This figure illustrates a comparison between the specificities resulted from AuNP wash, under the dynamic flow condition and under the stop-flow condition. The stop-flow AuNP wash was performed by incubation of the wash solution in the CD-NBA channels for 15 min. Although this AuNP wash has resulted in only a slightly higher specificity in comparison with the corresponding stringent wash (1.8 vs 1.2), the dynamic wash method leads to a much enhanced specificity (up to 3.9) within similar wash times (i.e. 700 rpm chip rotation takes ˜15 min.). We believe that the enhanced specificity is due to the flow-mediated convective mass transport in dynamic AuNP wash which is much more effective than the diffusion-mediated mass transport in stop-flow wash. The former method leads to higher effectiveness in AuNP-enhanced destabilization of MM duplexes, and hence, higher specificities.

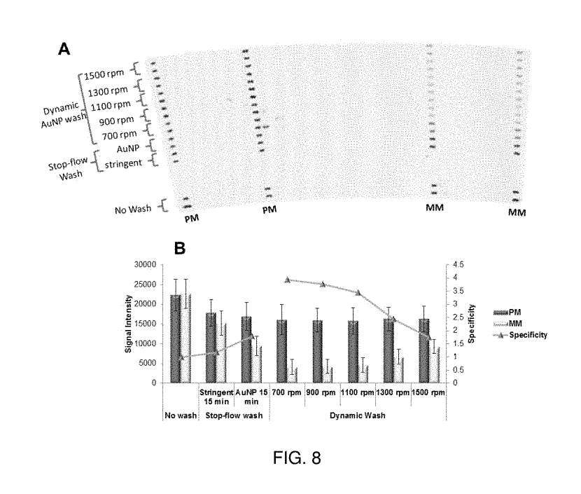

FIG. 9: The fluorescence image of CD-NBA chip with the patches resulted from dynamic DNA hybridization (spin rate of 900 rpm) of A20 target with its PM probe (A) and MM probe (W) at spin rate of 900 rpm and temperature of 22° C. After DNA hybridization, the hybridization patches were washed with 2 μL of SSC buffer with concentrations from 0.01× to 2× (i.e. NaCl concentrations from 1.5 to 300 mM, respectively) at 3 different temperatures of 22, 30 and 40° C. For AuNP wash, the SSC 1× buffer solution (consisting of 150 mM of NaCl) contained 5-nm AuNPs with various concentrations of 0.2-40 nM. In both wash methods, the flow of 2 μL of wash buffer was introduced by a spin rate of 900 rpm.

FIG. 10: The sensograms resulted from kinetic analysis of DNA hybridization by SPR spectroscopy. A20 target with 5 concentrations (10, 20, 40, 80 and 160 nM) were prepared in the HBS-N buffer and the hybridization was conducted at 22 and 40° C. The DNA hybridization step (60 s) were followed by the wash step (240 s). Either PM probes (A) or MM probes (W) were previously immobilized on the SPR sensor chip surface. The sensograms (a), (b) and (c) were resulted from the hybridization of A20 target with the PM probe (A) and the sensograms (d), (e) and (f) were resulted from the hybridization with the MM probe (W). During the wash step the duplexes were washed at 3 different conditions, i.e. in a flow of HBS-N 1× buffer solution without AuNPs at 22 and 40° C., and in a flow of the same buffer with 5-nm AuNPs (10 nM) at 22° C. The AuNPs were loaded with 10 nM of irrelevant oligonucleotides (20-mer). The hybridization rate constants (kₕ) and the dehybridization rate constants (k_{d}), resulted from kinetic analysis on the sensograms, are shown above the them. The standard errors (in parenthesis) are resulted from 2 measurements each performed with 5 different target concentrations.

FIG. 11: Signals obtained from hybridization of amplicons (PCR, HDA and nanoHDA) with their complementary probes. The probes (20-mer), which were immobilized in the NanoBioArray (NBA) chip, consisted of P-W (for wild-type KRAS) and P-PC (for positive control). FIGS. 11A and 11B show the scanned fluorescence images and the corresponding histograms, respectively. The target in lane 1 is a 102-bp amplicon from a bacterial DNA (b-DNA) sample (Neisseria gonorrhoeae), which serves as the positive control. The targets in lanes 2 to 4 are 92-bp HDA amplicons from a 162-bp gene fragment as the DNA template (lane 2), from human gDNA template without (lane 3), and with (lane 4) NlaIII restriction enzyme treatment (digest). Line 5 is a 92-bp PCR amplicon from human gDNA. Lanes 6 to 9 are the signals from 92-bp HDA amplicons: digest without AuNP (Lane 6), 5 nm AuNP (2 nM) without digest (Lane 7), 5 nm AuNP (2 nM) with digest (Lane 8) and 10 nm AuNP (0.2 nM) with digest (Lane 9) added to the HDA reagent. In all cases using the human gDNA, the primers employed were identical and the 92-bp amplicons with the same sequence of the wild-type KRAS gene was generated.

FIG. 12: A) The hybridization signals obtained from nanoHDA amplicons using different concentrations of 5 nm and 10 nm AuNPs in the HDA mixture. B) The hybridization signals from HDA and nanoHDA amplicons (using 10 nM of 5 nm AuNPs) at various concentrations of the HDA enzyme mix, i.e. 1× (1 μL), 2× (2 μL), 3× (3 μL), and 4× (4 μL). C) The capillary gel electropherograms of the PCR, HDA and nanoHDA amplicons prepared at different conditions. Primer concentrations in different amplification mixtures were 400 nM in PCR, 75 nM in HDA 1, nanoHDA 1 and nanoHDA 2, and 200 nM in HDA 2 and nanoHDA 3.5 nm AuNPs (6 nM) were used in nanoHDA 1 and nanoHDA 3, and 10 nm AuNP (0.6 nM) was used in nanoHDA 2. All amplification experiments generate the same 92-bp amplicon from human gDNA.

FIG. 13: A) The graphs show the signals from hybridization of HDA amplicons in the NBA chip. The amplicons were prepared using HDA and nanoHDA (5 nm, 6 nM) with the incubation times in the range of 0-120 min. The signal-to-noise ratio (SNR) of 10 (˜3300) was chosen as a minimum acceptable value of the signal intensity. B) histograms show the hybridization signals of the amplicons prepared using nanoHDA performed at temperature range of 40-65° C. C) SNP detection assay on nanoHDA amplicons in the NBA chip. The scanned image and the histogram were obtained from the hybridization of nanoHDA amplicons (1× enzyme mix, 1 h incubation at 65° C., 0.3 nM of 10 nm AuNP), with their corresponding perfectly matched (PM) and mismatched (MM) probes in the NBA chip. The target molecules (92 bp) were amplified from 4 different alleles of KRAS gene codon 12 and hybridized with their complementary probes preprinted on the chip surface. Each probe is perfectly matched with one of the targets and single base-pair mismatch with the other 3 targets. After hybridization, washing was conducted with a flow of hybridization buffer (15 mM sodium citrate with 90 mM NaCl) containing 5 nm AuNPs (5 nM, stabilized with 8 nM of 12mer irrelevant oligonucleotides) at a temperature of 22° C. for 10 min.

### DETAILED DESCRIPTION

Throughout the following description, specific details are set forth in order to provide a more thorough understanding of the invention. However, the invention may be practiced without these particulars. In other instances, well-known elements have not been shown or described in detail to avoid unnecessarily obscuring the invention. Accordingly, the specification and drawings are to be regarded in an illustrative, rather than a restrictive, sense.

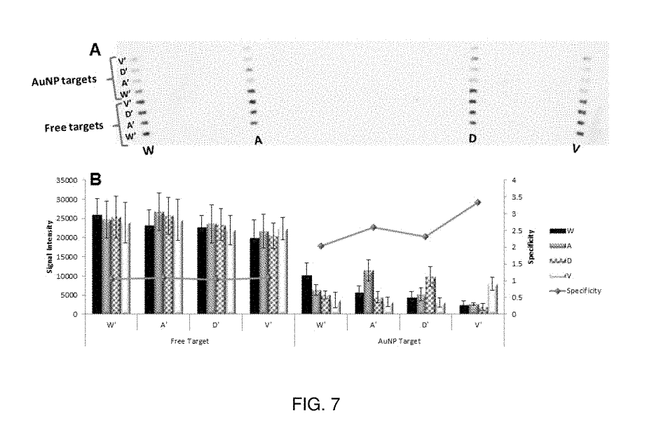

A microfluidic bioarray technique has been developed, and this technique uses gold nanoparticles (AuNP targets) for specific detection of single nucleotide polymorphism (SNP). In this technique, no temperature stringency is required, and high specificities in hybridization are achieved by loading the target strands on the crusts of small gold nanoparticles (AuNPs) prior to their hybridization to the oligonucleotide probes immobilized on the microfluidic channel surfaces. Our kinetic studies of DNA hybridization using surface plasmon resonance (SPR) spectroscopy has showed that AuNPs enhanced the dehybridization of the mismatch (MM) duplexes more than that of perfectly matched (PM) duplexes, thus accounting for most of the SNP discrimination power of the AuNP-enabled technique. However, the AuNP targets result in lower hybridization signal intensities than the free target counterparts (See FIG. 7).

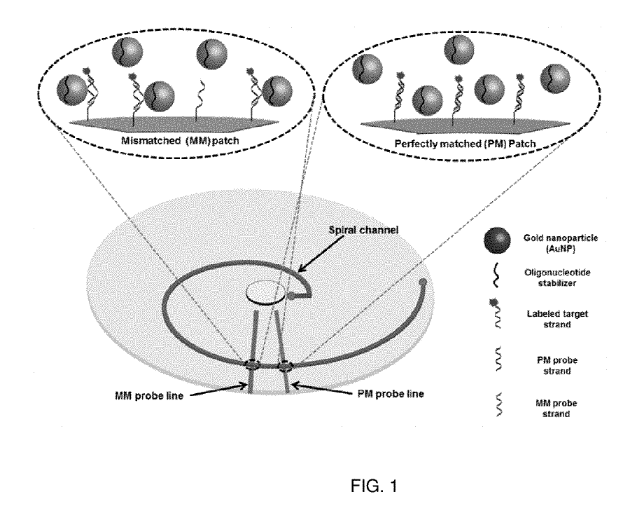

As inspired from our kinetic analysis, we know that the influence of AuNPs is predominantly on dehybridization. Therefore, we attempt to develop a method to use AuNPs in the washing (dehybridization) step, rather than using them in the hybridization step. In this method, a buffer solution containing AuNPs (5-nm diameter or some other suitable size) is used to flow over the surface-bound duplexes for the removal of the hybridized oligonucleotides by washing (FIG. 1). The AuNP-enhanced dehybridization is achieved via targeted binding between AuNPs and the thermally induced openings along the DNA duplexes. The AuNP-ssDNA interactions stabilize the openings, and thus accelerate their propagation, which in turn accelerate dehybridization and preferentially destabilize the MM duplexes.

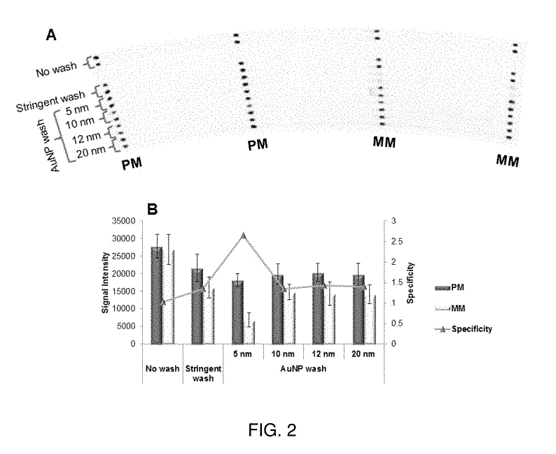

For nucleic acid analysis, we have previously developed a CD-like chip for microfluidic DNA hybridization that provides the advantage of fast analyses and multiplex capability. This platform, termed as CD-like NanoBioArray chip or CD-NBA chip, utilizes the centrifugal force in order to flow in the target solutions within the microfluidic channels. As shown in FIG. 1, the target molecules hybridize to their complementary probes located at the intersections of the spiral channels to the radially-patterned probe lines. These probe lines have previously been printed on the surface of the chip, and hybridization occurs between the biotin-labeled target DNA molecules and complementary DNA probes, giving rise to fluorescence hybridization patches. FIG. 2A shows the fluorescence image of the hybridization on a region of the CD-NBA chip, in which several spiral channels intersect with four probe lines. After DNA hybridization, different types of wash were applied in the spiral channels. The histogram of the fluorescence intensities of the patches is shown in FIG. 2B, with the specificity (σ) also shown in Eq. 1 as follows.

$$σ = \frac{{S}_{pm}}{{S}_{mm}} \qquad ( 1 )$$

where Sₚₘ and Sₘₘ are signal intensities at the PM and MM patches, respectively. In DNA microarrays, the nonspecific signals are inevitably detected and they are conventionally reduced by conducting a stringent wash subsequent to DNA hybridization. To compare the methods of AuNP wash and stringent wash, we flow the hybridization buffer (SSC 1×) in the spiral channels at room temperature, with or without AuNPs, respectively. The stringent wash only results in a specificity of 1.3 (compared to 1 in “no wash” channels). On the other hand, the use of AuNPs in the wash step helped to improve specificity to 2.6 but it was only in the presence of AuNPs of 5 nm, but not of 10, 12 and 20 nm, in diameter that the specificity was enhanced by washing (˜2.6). This result is in agreement with the previous observation, in which AuNPs have been used in the hybridization step.

### Signal/Specificity Correlation in AuNP Wash Technique

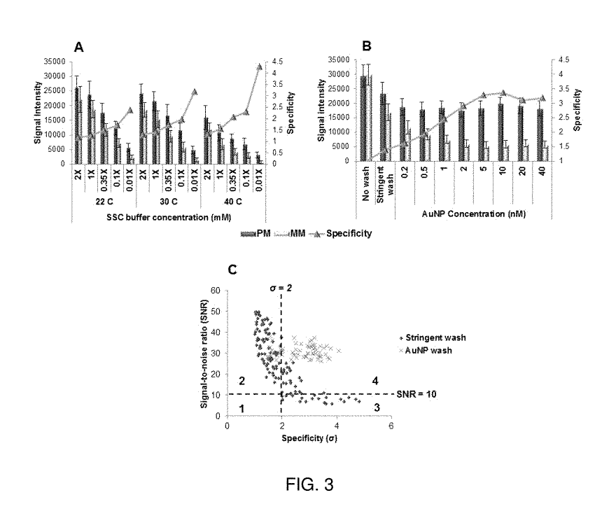

In the stringent wash method, high-temperature or/and low-salt conditions are used to create a destabilizing environment for the formed duplexes and accelerate their dehybridization. This method aims to remove the nonspecific duplexes more than their specific counterparts, thus enhancing the specificity. We compare the AuNP wash and the stringent wash methods directly. FIGS. 3A and 3B show the histograms resulted from the hybridization signals following stringent wash and AuNP wash, respectively. FIG. 3A indicates that the specificity increases as the level of stringency increases (i.e. higher temperature and less salt), but the PM signal undesirably decreases too, resulting in a negative correlation (anticorrelation) between the signal and specificity. We employed the Pearson correlation coefficient (r) as a measure of correlation of the PM signal (S_{PM}) and specificity (□), see Eq. 2 as follows,

$$r = \frac{{\sum }_{i = 1}^{n} 〚 {( S 〛}_{PM}^{i} - {\overline{S}}_{PM} ) ( {σ}^{i} - \overline{σ} )}{\sqrt{{\sum }_{i = 1}^{n} {〚 {( S 〛}_{PM}^{i} - {\overline{S}}_{PM} )}^{2}} \sqrt{{\sum }_{i = 1}^{n} {( {σ}^{i} - \overline{σ} )}^{2}}} \qquad ( 2 )$$

where Sₚₘ is the PM signal intensities at different washing condition; S̅_{PM} is the average intensity for PM signals; [symbol] is the specificity (calculated by Eq. 1) at each washing condition; σ̅ is the average specificity; n is the number of data points.

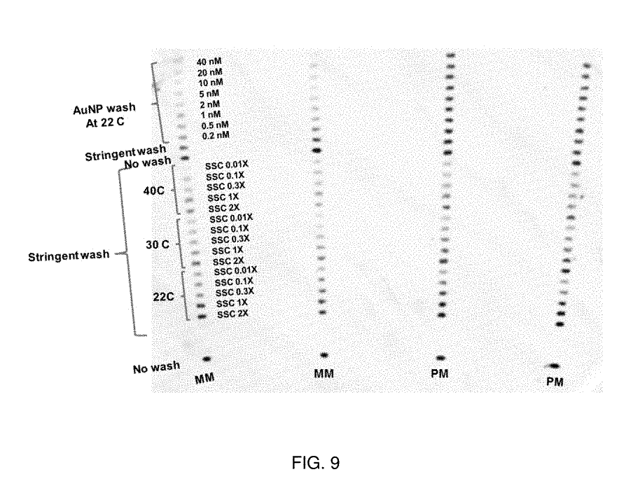

While r=0 shows no correlation or the situation when specificity is achieved without a loss in signal, r=−1 shows the highest anticorrelation between the signal and specificity. From the signals and specificities shown in FIG. 3A, the r value is determined to be −0.92 (For details, see FIG. 9), which indicates a strong anticorrelation between the two parameters. Unfortunately, this anticorrelation between signal and specificity is frequently reported in DNA hybridization experiments using the stringent wash method, and high specificity appears to only be achieved at the expense of the signal. On the other hand, washing of the duplexes using buffer solutions carrying AuNPs do not display such a strong anticorrelation. FIG. 3B shows the hybridization signals after washing the duplexes with the hybridization buffers containing AuNPs at various concentrations. The MM signals decreases as the AuNP concentrations increase from 0.2 to 5 nM but no further decrease is observed at higher AuNP concentrations (5-40 nM). Since the PM signals are not reduced with the increasing AuNP concentration, this leads to a maximum specificity of 3.2 at 5 nM AuNP. The calculated r value for the AuNP wash method is −0.16, which indicates a much lower signal/specificity anticorrelation, or almost no correlation, obtained from this method, in comparison with the value of −0.92 obtained from high-temperature/low-salt stringent wash method.

The difference between the r values obtained from AuNP wash and stringent wash is also illustrated in our analysis of ˜400 hybridization patches obtained by both methods. FIG. 3C shows a plot of signal-to-noise ratio (SNR) vs. specificity (σ) obtained from AuNP wash and stringent wash. We defined the minimum acceptable values for SNR as 10 and for σ as 2. We also divided the plot into 4 regions of low σ/low SNR (region 1), low σ/high SNR (region 2), high σ/low SNR (region 3) and high σ/high SNR (region 4). Obviously, the majority of the data points resulted from stringent wash are distributed in regions 1-3. It was found that the data points from the AuNP wash method are primarily localized in region 4, which correspond to the desirable outcome of high σ and SNR.

The outcome of high σ and minimal loss in SNR observed with the AuNP wash method can be explained in terms of the dehybridization rate constant k_{d}, which is experimentally determined from our kinetic analyses using SPR spectroscopy. As shown in Table 1, for the MM duplex the k_{d} value (in 10⁻⁴ s⁻¹) is observed to enhance by five times, i.e. from 3.2 for stringent wash to 15.9 for AuNP wash at 22° C. On the other hand, the k_{d} value (in 10⁻⁴ s⁻¹) for the PM duplex has not increased much, i.e. from 1.7 for stringent wash to 3.0 for AuNP wash. This increase in the k_{d} value for AuNP wash (less than two-fold) is much smaller than the corresponding increase for 40° C. stringent wash (five-fold). This observation is attributed to the enhanced dehybridization of the MM duplexes by AuNPs. On the other hand, increasing the stringent wash temperature from 22° C. to 40° C. enhanced the k_{d} values of both MM duplexes and of PM duplexes, showing the undesirable destabilization of PM duplexes, in addition to the desirable destabilization of MM duplexes. These observations explain our findings obtained in the CD-NBA chip that the PM signals are not affected as much as the MM counterparts in the AuNP wash method, because of the enhanced destabilization of the MM duplex, but not of the PM duplex, leading to the preservation of the signal.

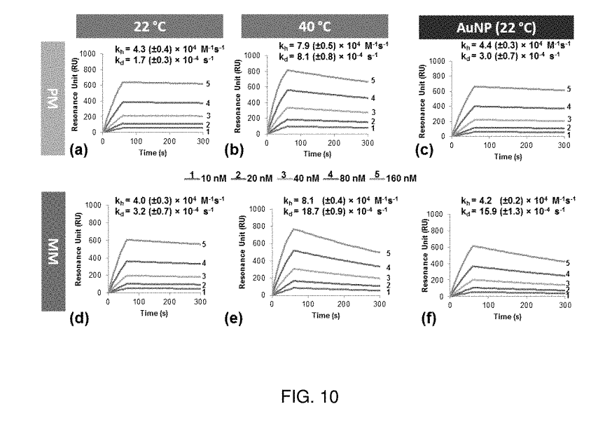

TABLE 1
Dehybridization rate constants (k_{d}) of PM duplexes
and MM duplexes using stringent wash and AuNP wash,
as determined from SPR spectroscopy (See FIG. 10).

| | |
|---|---|
| Stringent wash | AuNP Wash |

| | | 22° C. | 40° C. | 22° C. |
|---|---|---|---|---|
| k_{d}/ | PM | 1.7 (±0.3)^{a} | 8.1 (±0.8) | 3.0 (±0.7) |
| (10⁻⁴ s⁻¹) | MM | 3.2 (±0.7) | 18.7 (±0.9) | 15.9 (±1.3) |

^{a}All standard errors are determined from two measurements each including five different target concentrations of 10, 20, 40, 80 and 160 nM.

We attribute the difference in enhanced destabilization of the MM duplexes, observed for AuNP wash, compared to stringent wash, to the specific mechanism on which the AuNP wash technique is based. During dehybridization, AuNPs bind to the ssDNA segments (bubbles), which have constantly formed by thermal breathing. The presence of a mismatch base pair, through a cooperative effect, causes weakening and disruption of the neighboring base pairs. In 2006, Zeng and coworkers compared the dissociation curves obtained from PM and MM duplexes, and found that the amount of bubbles was drastically enhanced in the presence of a single MM site in the middle of the duplex. The greater amount of bubbles in the MM duplexes makes them susceptible to the binding by AuNPs, leading to the success of the AuNP wash method. The AuNP wash method target these bubbles in MM duplexes for their enhanced dehybridization or destabilization, to a much larger extent than in the case of PM duplexes. This targeted mechanism of destabilization of MM duplexes causes an enhancement in the specificity without reducing the signal, leading to the observed low negative r value or almost no anticorrelation between signal and specificity. On the other hand, the stringent wash method has similar destabilizing influences on both of the PM and MM duplexes, which lead to their similar extent of accelerated dehybridization and the observed high signal/specificity anticorrelation, or high negative r value.

The preserved sensitivity upon enhancement of specificity is an exclusive feature of the AuNP wash method. This feature was not achieved in the previous AuNP-enabled method, in which AuNP was used in the hybridization step but not in the wash step. In the previous method, the hybridization signals obtained from DNA targets that are conjugated to AuNPs (AuNP targets) were observed to be lower than the signals from free targets, and this observation is attributed to the low hybridization rate constants (kₕ) of DNA targets, when conjugated to AuNPs. The experiment has been repeated in the CD-NBA chip and shown in FIG. 7.

### Optimization of the AuNP Wash Method

In order to optimize the AuNP wash method, we evaluate the effect of different experimental factors including the salt content of the buffer medium, the length and concentration of the oligonucleotide stabilizer (used to prevent AuNPs in the wash buffer to aggregate) on the performance of the method. Optimization of these factors can improve the effectiveness of AuNP destabilization of MM duplexes, and thus the efficacy of the method.

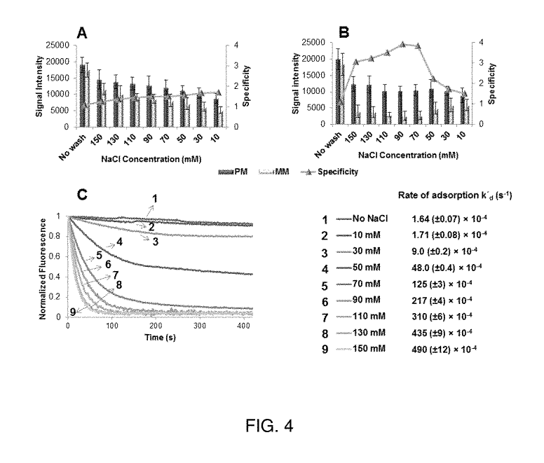

The histogram in FIG. 4A shows the hybridization signals after stringent wash. As the salt concentration is reduced, the signal decreases, and the specificity increases. This signal/specificity anticorrelation is consistent with the results in FIG. 3A, which displayed data at narrower range of salt concentrations, though at several temperatures. The histogram in FIG. 4B shows the signals of hybridization after washing with the buffer solutions containing 5 nM of AuNPs (5-nm AuNP, 150 mM NaCl). FIG. 4B displays a similar increasing trend for specificity with reducing salt contents from 150 to 90 mM, but a different trend, now decreasing, at lower salt contents (from 50 to 10 mM of NaCl) reaching the specificities comparable to the values obtained from stringent wash. This latter trend indicates that the AuNPs become ineffective in the destabilization of the MM duplexes at low salt concentrations. We attribute this ineffectiveness to the low extent of binding between AuNP crusts and ssDNA segments of the duplexes (bubbles) at low salt concentrations. To prove this low rate of binding, we measure the adsorption kinetics of ssDNAs onto the crusts of AuNPs at different salt concentrations. This measurement is based on the fact that the emission of the fluorescently-labelled DNAs is quenched after they bind to AuNPs. FIG. 4C shows the kinetic traces of the normalized fluorescence of a fluorescently labelled 20-mer oligonucleotide upon mixing with AuNPs at different NaCl concentrations. The pseudo first-order rate constant of adsorption of oligonucleotides onto the AuNP crusts, k′_{d}, was obtained from the exponential fit of the kinetic data (See FIG. 4C). The k′_{d} values increase from 1.64×10⁻⁴ s⁻¹ at no-salt condition to 490×10⁻⁴ s⁻¹ at 150 mM of NaCl. This increasing trend of k′_{d} values with salt concentrations may be explained by the fact that electrostatic repulsion between the negatively charged DNA backbone and the citrate-capped crusts of AuNPs is reduced by charge screening at high salt concentrations [29].

Using the data in FIG. 4C, we can explain the salt-dependency of the AuNP-enhanced destabilization, and of the specificities, that are observed in FIG. 4B. First, an increase in the salt content enhances the AuNP-ssDNA binding, due to charge screening effect on AuNPs and ssDNAs. This effect enhances the effectiveness of the AuNP wash method, and thus the specificity. The sharp enhancement in the specificities resulted from the AuNP wash method at NaCl concentrations of 30 to 70 mM (FIG. 4B) indicates that the charge screening of AuNP-ssDNA prevails at this range of salt content. Second, salt also decrease the specificities through charge screening of probe ssDNAs and target ssDNAs. This phenomenon is similarly observed in AuNP wash (FIG. 4B) and stringent wash (FIGS. 3A and 4A). The decreasing trend of specificities at high salt concentrations (90 to 150 mM NaCl) shows that, at this range of salt content, the increase in the AuNP-ssDNA binding is less effective than the increase in the probe-target binding.

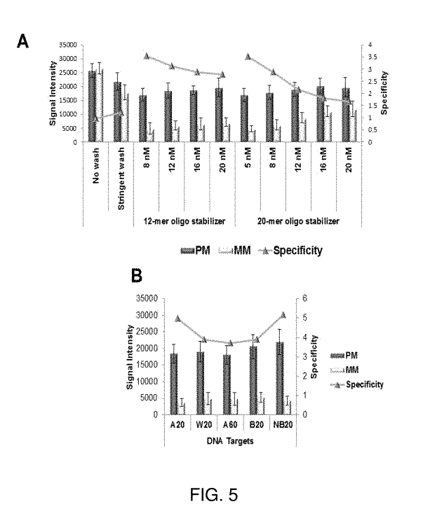

In order to stabilize AuNPs in the wash buffer against salt-induced aggregation, the AuNP crusts have been loaded with oligonucleotide stabilizers with sequences non-complementary to the probe/target sequences. The aggregation would have happened to the pristine nanoparticles due to high salt contents in the wash buffer. Here, we investigate the effects of length and concentration of the oligonucleotide stabilizers on the specificities obtained in the AuNP wash method. FIG. 5A shows the hybridization signals after the duplexes were washed with solutions containing AuNPs that have been stabilized with 12-mer and 20-mer of irrelevant oligonucleotides of different concentrations. It is observed that higher specificities are obtained when shorter oligonucleotides (12-mer rather than 20-mer) and/or lower concentrations of oligonucleotide are used. The specificities are higher maybe because the oligonucleotide stabilizers with shorter sequence lengths and lower concentrations occupy smaller portions of the AuNP crusts, and thus leaving greater portions available for binding to the ssDNA segments of the duplexes. Additionally, since the negative charges of the oligonucleotides add to the negative charge density on the AuNP crusts and hinder AuNPs from approaching, and attaching to, the duplexes, which are also negatively-charged, shorter lengths and lower concentrations of the oligonucleotide stabilizer will lead to a higher effectiveness in AuNP-enhanced destabilization of MM duplexes, and to higher specificities.

### Applications for Genomic Samples

In order to investigate the applicability of the AuNP wash technique for use with genomic samples, we first evaluated the robustness of the technique upon sequence variation (i.e. the purine content), and then we evaluated the performance of the technique using PCR amplicons as the target strands.

In order to evaluate the robustness of the AuNP wash technique, we employ 3 sequences related to KRAS gene (A20, A60, W20), and two sequences related to a fungal pathogen (B21, NB21); see Table 2. In W20 and A60 targets, the 20 bases of the target that hybridize with the probes are similar to A20 except for variations in the type of the mismatch base-pair (C-C base-pair in A20 and A60 vs. G-G in W20) and also in the length of the target (60 bases in A60 vs. 20 bases in A20). As shown in FIG. 5B, these sequence variations do not affect the performance of the technique. Experiments were also performed using sequences that are completely different from A20, i.e. B21 and NB21. The strength of binding with gold is known to vary among DNA bases, and purine bases (A and G) are known to bind more strongly with gold than pyrimidine bases (C and T) [30]. Since B21 and NB21 targets have lower purine base contents in their sequences, in comparison with the A20 target (˜40% in B21/NB21 targets vs. 60% in A20), we expect to observe lower specificities among these B21 and NB21 targets. However, as observed from FIG. 5B, the lower purine base content of B21/NB21 targets does not result in a decrease of the specificities. Since either the binding of AuNPs to the target strand or the binding to the probe strand can accelerate the dehybridization process, the weaker binding between AuNPs and the pyrimidine-rich strand offset the stronger binding between AuNPs and the complementary purine-rich strand. This offset effect leads to an insensitivity of the AuNP wash method to the purine content of the DNA sequence. With the robustness of the method demonstrated, we conclude that the AuNP wash method can be applied to hybridization experiments involving DNA strands with various sequences.

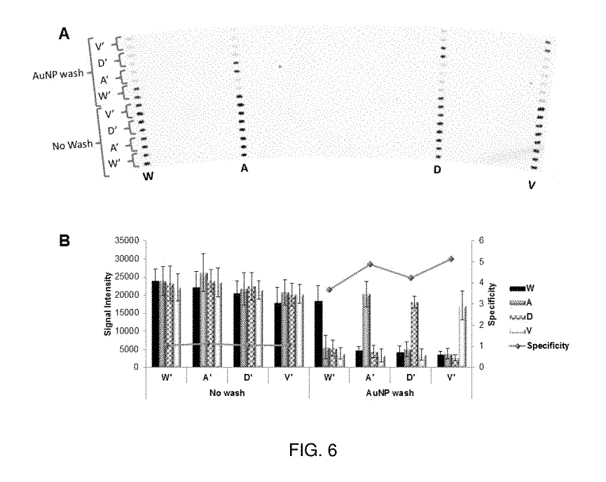

We have also used the AuNP wash method to detect single nucleotide polymorphisms (SNPs) in genomic samples, which consists of 4 different alleles of KRAS gene codon 12. The detection of these SNPs is critical for clinicians to choose the appropriate type of therapy for colorectal cancer patients [31]. FIG. 6A shows the fluorescence image of the signals obtained from the PCR amplicons that have been hybridized to the probes on the surface of CD-NBA chip followed by AuNP wash. As displayed in FIG. 6B, the specificity was enhanced without compromising the signal, leading to a sensitive and specific SNP discrimination obtained at ambient temperature (22° C.). These results, obtained by using AuNPs in the wash solution, are in sharp contrast with the previous results in FIG. 7 obtained by using AuNPs that have been conjugated to the DNA targets in the hybridization solution. This is because the sensitivity of the PM duplexes in the current AuNP wash method is preserved while the specificity is enhanced.

We have developed a technique for the enhancement of the specificity of DNA hybridization without reducing the signal. This technique is called AuNP wash, which may be performed in a CD-NBA chip using a buffer solution containing 5-nm gold nanoparticles (AuNPs). The solution dynamically washes the duplexes on the surfaces of the spiral channel of the chip and destabilizes the MM duplexes but not the PM duplexes. The nanoparticle does not bind to the fully coiled duplex, but does only target the ssDNA segments (bubbles) of the duplex in the course of dehybridization and accelerate the propagation of the bubbles and unzipping of the duplex. This mechanism of destabilization causes a preferential removal of the MM duplexes, rather than the PM ones, and hence the signal is preserved, while the specificity is enhanced. We have also studied the influence of several governing factors of the method, evaluated the performance of the technique upon the variation of the DNA sequences, and applied the method for detection of KRAS gene SNP variations in genomic samples. Furthermore, the SNP discrimination is achieved at a single temperature, alleviating the difficulty of temperature optimization for multiple targets of different melting temperatures in multiplex analysis. In contrast to the other attempts (e.g. molecular beacons) to enhance the specificities of DNA hybridization, no complicated design for the DNA probe sequence is required and high specificity is effectively achieved via a simple wash step subsequent to DNA hybridization. This simplicity is an advantage which, together with the robustness upon sequence variation and compatibility with multiplex analyses, makes this technique a promising tool to be used in DNA hybridization-based microarrays with the potential to reduce false positive/false negative results and improve the accuracy of the microarray results.

Other than hybridization, for DNA amplification we have developed the nanoparticle-assisted helicase-dependent amplification (HDA), termed nanoHDA, by enhancing the efficiency of conventional HDA using AuNPs. The nanoHDA technique is then coupled to our AuNP-enhanced technique for detection of SNPs in the KRAS gene. To the best of our knowledge, this is the first report on the use of nanoparticles for improving an isothermal amplification technique.

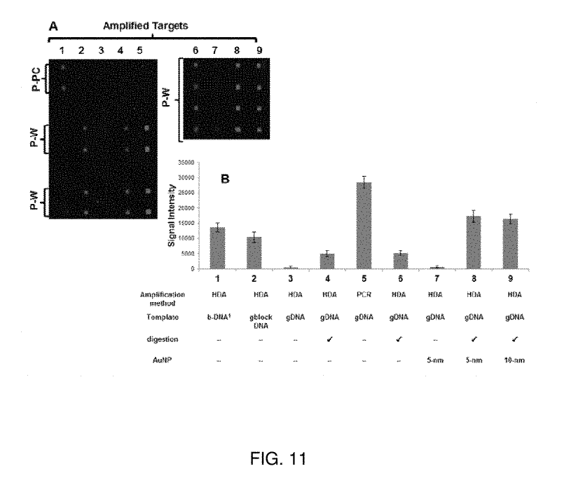

FIG. 11 shows the fluorescence images and the corresponding histogram obtained from DNA hybridization between HDA and PCR amplicons with complementary oligonucleotide probes immobilized on the surfaces of NBA chip channel. Lane 1 shows, in duplicate, sufficient intensity of the patches formed by the surface hybridization of a 102-bp HDA product amplified from a bacterial DNA template (b-DNA), which is served as the HDA positive control. The intensity of the hybridization patches in lane 2 obtained from the HDA amplicon based on a 161 ssDNA template is also sufficient, indicating the success of the HDA method on amplifying the KRAS sequence. However, the signal in lane 3 obtained from the HDA amplicon generated from the human gDNA template is very low. On the other hand, lane 5 shows a strong hybridization signal obtained from the PCR amplicon using the same gDNA template identical to the one used in producing the HDA amplicon shown on lane 3. These results, also shown in the histogram, indicate sufficient hybridization intensities to conclude the observations that HDA has successfully amplified the ssDNA template but not the gDNA template, and the latter was successfully amplified by PCR.

We attribute low HDA signals on lane 3 to the low efficiency of helicases to denature long dsDNA templates, which may cause HDA to fail in amplifying long gDNA, but not short ssDNA. In contrast, PCR is successful (lane 5) because it denatures the template by heating, which is capable of quickly denaturing even long dsDNA. To overcome the issue of DNA length, we treated the gDNA template with the restriction enzyme NlaIII (New England Biolabs) to generate DNA fragments of reduced lengths. This restriction enzyme was chosen to perform a digestion and create a 240-bp fragment which contains the KRAS sequence. Lanes 4 and 6 show the results of improved intensities for the HDA amplicons obtained from the 240-bp restriction fragment. These results are consistent with the previous report by Tong et al. that the use of a restriction enzyme as an additive in the HDA reagents improved the amplification of a bacterial DNA [29].

These improved results in lanes 4 and 6 also confirm our hypothesis that the HDA efficiency for long DNA templates is low because of the limited capability of helicase-mediated template denaturation. However, the improved signal is still not comparable to the signal obtained from the HDA amplicons generated from the 162-gene fragment template (lane 2). As inspired from the use of nanoparticles in PCR, we added gold nanoparticles (AuNPs) in the HDA reagents to assist in the helicase-mediated denaturation of templates, a new method we dubbed nanoHDA. The hybridization signals in Lanes 8 and 9, which were obtained from HDA on gDNA with AuNP added to the amplification mixture. Even when AuNPs were used, the template should still be digested with the restriction enzyme, as seen from the low intensity in Lane 7 when only AuNP but not restriction enzymes was used. A comparison between the signals in lanes 8 and 9 shows that the use of different sized AuNPs (5 nm, 10 nm, respectively) has a similar enhancing effect on HDA. On the basis of these results, we speculate two ways that AuNPs assist helicases in dsDNA denaturation and thus enhance HDA. First, AuNPs may have a preferential affinity for ssDNAs versus dsDNAs. Thus in a similar fashion to that of single-stranded binding (SSB) protein, nanoparticles may bind to the ssDNA segments and prevent them from renaturation, which assists the helicase-dependent denaturation. Second, AuNPs may be able to directly affect the dsDNA segments and enhance their denaturation [21, 58, 59], a capability that has not been reported for SSB protein. Once bound to a partially denaturated DNA, AuNPs destabilize the neighbouring base-pairs and accelerate denaturation of the dsDNA segments.

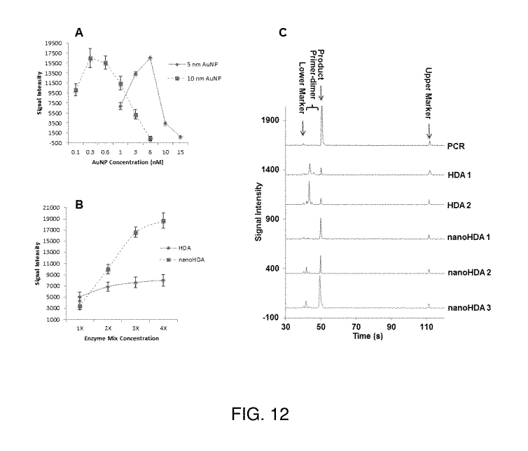

To examine if a greater number of AuNPs enhance HDA even more, we studied the effect of different amounts of AuNPs on nanoHDA. FIG. 12A shows the hybridization signals obtained from the HDA amplicons prepared using different concentrations of AuNPs (with diameters of 5 nm and 10 nm), suggesting the yield of amplification increases with the addition of AuNPs, but only to an optimum concentration (6 nM for 5 nm and 0.3 nM for 10 nm AuNPs). Thereafter, the yield decreases to a low signal at 15 and 6 nM for 5 nm and 10 nm AuNPs, respectively, due to HDA inhibition. These inhibitory results on HDA are consistent with the results reported for PCR [52, 55, 60, 61], but higher AuNP concentrations were involved in HDA than in PCR. Such a higher AuNP concentration may be caused by greater crust area, as the complete HDA inhibition for 25 μL of HDA mix occur at total nanoparticle crust areas of 17.5 and 18.7 mm² for 5 nm and 10 nm AuNPs, respectively, which are higher than the corresponding values of 3.7 mm² [55], and 12 mm² [35, 60], reported for PCR. We think the higher tolerance of HDA to inhibition due to high concentrations of AuNPs, as compared to PCR, might be caused by a higher concentration of dATP (3 mM vs. 0.2 mM) used in HDA than in PCR, as dATP is used both as DNA building blocks and as a cofactor of helicase [62]. Since dATP is known to have the strongest affinity for AuNP surfaces among nucleotides [63], it may block the nanoparticle crusts, reduce the adsorption of HDA enzymes on the AuNP crusts, thus allows HDA to proceed at higher AuNP concentrations.

To confirm if HDA inhibition at high AuNPs concentration is due to a loss of HDA enzymes on the nanoparticle crusts, different enzyme mix concentrations were used and the results were compared. As shown in FIG. 12B, at an enzyme mix concentration of 1×, the signal intensity due to hybridization of the amplicons obtained with nanoHDA (˜3400) is significantly lower (p<0.05) than the corresponding signal of HDA (˜5000), which indicates partial inhibition due to AuNPs (5 nm, 10 nM) in nanoHDA. This AuNP-induced inhibition was also observed in FIG. 12A when 10 nM of 5 nm AuNP was used in the nanoHDA mix. On the other hand, the hybridization signal intensities increase when the enzyme concentration increases from 1× to 4× for both amplification techniques. In addition, a significantly higher increase is evident for nanoHDA as compared to HDA (increases of 240% and 37%, respectively, when 4× enzyme mix was used). This observation confirms that the inhibition due to loss of enzymes on AuNP crusts can be compensated by using a higher concentration of enzymes, which lead to signal intensities that cannot be achieved in the HDA method even with a higher enzyme concentration.

NanoHDA also reduced the nonspecific amplification of primer-dimers. Their formation is evidenced in the results of the primer-dimer peaks obtained using capillary gel electrophoresis (CGE). As shown in FIG. 12C, a comparison between the electropherograms of HDA product mix (HDA1) and PCR product mix (PCR) shows a large peak of primer-dimer in HDA1 but not PCR, and the peak height of the 92-bp product is reduced in HDA1. Although identical primer set and annealing temperature (65° C.) were used for both DNA amplification, PCR benefited from thermocycling to reach higher temperatures due to elongation (72° C.) and denaturation (94° C.), which had reduced the formation of primer-dimers. A higher amount of primer used in HDA2 (200 nM), as compared to HDA1 (75 nM), resulted in the formation of an even larger primer-dimer peak, which explained why lower primer concentrations is typically used in HDA (75 nM), as compared to PCR (400 nM). The electropherograms also show that the addition of nanoparticles resulted in smaller primer-dimer peaks and hence larger product peak, compared to HDA without AuNP (see nanoHDA2 versus HDA1, or nanoHDA3 versus HDA2). The results of reduction of primer-dimer formation are even better when 5 nm AuNPs were used (see nanoHDA1 versus nanoHDA2). This effect of AuNPs in reducing the formation of nonspecific products is caused by the interaction of DNA bases with AuNP crusts, which is consistent with our previous observations on DNA hybridization conducted in an NBA chip [19, 21]. We believe that a similar interaction reduced the formation of primer-dimers, thus contributing to the enhancement of the HDA product formation. Such a nanoparticle-assisted mechanism is further demonstrated in the effect of the amount of primers used in nanoHDA. When comparing nanoHDA 3 and 1, a higher amount of primers used in nanoHDA3 increased its product yield, which is in contrast to the comparison of HDA2 to HDA1, where a higher amount of primers used in HDA2 decreases its product yield. This difference is most likely due to the fact that nonspecific amplification is diminished by nanoparticles in nanoHDA, and so the increased primer concentration enhances the formation of amplicons by nanoHDA.

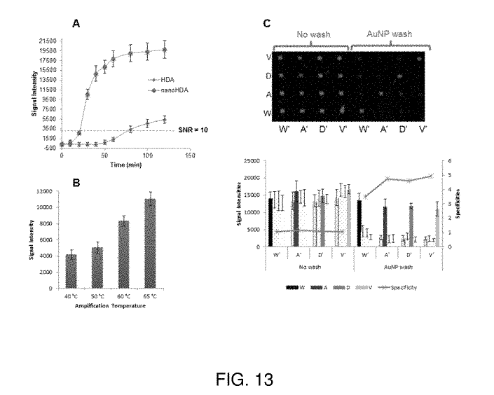

A comparison between the kinetics of HDA and nanoHDA was also conducted on the NBA chip to further understand the effect of nanoparticles on enhancing HDA. FIG. 13A shows the hybridization signals obtained using HDA and nanoHDA performed for periods of 10 to 120 min. A comparison between the two curves of fluorescent signal versus time shows that the hybridization signal for nanoHDA and HDA reaches a minimum acceptable signal-to-noise ratio (SNR) value of 10 at ˜20 min and ˜80 min, respectively, which indicates that a significantly faster amplification kinetics was obtained when induced by nanoparticles in nanoHDA. This observation can be explained in the light of our understanding that the rate-limiting step of HDA is the helicase-dependent denaturation, a process that can be facilitated by AuNP-ssDNA binding, resulting in an enhanced amplification.

A property resulted from the interaction between DNA bases and nanoparticles is used to enhance the reaction efficiency of PCR, as reported by several groups, and different mechanisms were proposed for this enhancement effect [51, 52]. First, Li et al. suggested that the preferential binding of single-stranded DNA (ssDNA) to AuNP surfaces, in a manner similar to single-strand binding protein (SSB), increased the specificity and sensitivity of PCR [53]. Second, the excellent heat-transfer property of nanoparticles is proposed to have shortened the reaction time for PCR [54], but this idea was later criticized by others [55]. Furthermore, Mi et al. suggested that AuNPs modulated the polymerase activity and enabled a hot start-like effect that suppressed nonspecific amplification at low temperature [56]. As inspired by these reports, we try to further enhance our nanoHDA technique by reducing the temperature from 65° that is used in conventional thermophilic HDA. However, we observe a significant signal reduction as the nanoHDA temperature decreases from of 65° to 40° (See FIG. 13B). We believe that these conflicting observations originate from the fundamental differences between isothermal HDA and PCR that uses thermocycling. Thermocycling allows for optimization of annealing and extension temperature separately, thus a change in the annealing temperature will not alter the extension temperature which affects the performance of the polymerase. On the other hand, annealing and extension in isothermal HDA occur at the same temperature, which prevents an independent optimization of the two temperatures. As the HDA temperature moves away from the optimum temperature for Bst polymerase (65° C.) the polymerase performance decreases, and so does the signal.

The nanoHDA technique provides an efficient platform for amplification of human genomic DNAs for subsequent hybridization-based detections. Therefore, we aim to combine nanoHDA with our AuNP-wash method to enable SNP detection using nanobioarray (NBA) chips. This combination allows for high-throughput SNP genotyping of the human genome. In the combined method, the amplicons were first prepared by a 1-h amplification using nanoHDA (10 nm AuNP, 0.3 nM) from 1 ng of gDNA with different alleles of KRAS gene. Thereafter, the HDA amplicons hybridized to an array of surface-bound oligonucleotide probes on the surface of an NBA chip, and finally the mismatched DNA duplexes were removed using the AuNP-wash method. As indicated by high sensitivity and specificity of the signals in FIG. 13C, this technique is successfully applied for SNP assays on the human gDNA samples.

In conclusion, we used gold nanoparticles (AuNPs) to improve sensitivity and specificity of helicase-dependent amplification (HDA). Our results show that preferential binding of nanoparticles to ssDNA facilitates helicase-mediated DNA denaturation and hence accelerates HDA and improves amplification sensitivity. In the presence of nanoparticles, the formation of primer-dimers were also suppressed which contributed to the high specificity of the technique. Finally, we successfully demonstrated SNP detection on human gDNA samples by coupling the nanoHDA technique with the AuNP-enhanced hybridization technique.

### Experimental Section

### Materials

Gold nanoparticles (with citrate and tannic acid) of 5-, 10- and 20-nm diameter were purchased from Sigma Life Science and 12-nm diameter gold nanoparticles (capped with citrate) were obtained from NanoComposix (San Diego, Calif.). Sodium dodecyl sulphate (SDS), 3-aminopropyltriethoxysilane (APTES), 25% glutaraldehyde, cetyltrimethylammonium bromide (CTAB) and Triton X-100 were purchased from Sigma-Aldrich. Negative photoresist (SU-8 50) and its developer were purchased from MicroChem Corp. (Newton, Mass.). Circular glass chips with 4-in. diameter and a 0.6-in. centre hole were obtained from Precision Glass & Optics (Santa Ana, Calif., USA).

All the reagents and materials required for surface plasmon resonance (SPR) experiments including 1-ethyl-3-(3-dimethylaminopropyl) carbodiimide (EDC), N-hydroxysuccinimide (NHS), ethanolamine, HBS-N Buffer (0.01 M HEPES pH 7.4, 0.15 M NaCl) and CMS sensor chips, were provided by GE Healthcare (UK).

All oligonucleotides (listed in Table 2) were synthesized and modified by Integrated DNA Technologies (Coralville, Iowa). Target oligonucleotides (20- or 60-mer) representing different SNPs of KRAS gene codon 12 (G12A (A) and wild-type (W)) and also 20-mer of B and NB targets (fungal pathogenic sequences [32]) were modified with a biotin molecule at the 5′-end. The probe sequences were designed in such a way that the SNP sites were located at the centre of the oligonucleotides. The 20-mer probe oligonucleotides were modified with an amine group and a C12 spacer at the 5′-end.

TABLE 2
The sequences of probe, target and primer oligonucleotides. The
underlined region of A60 is identical to the sequence of A20. The underlined
sequence of A60 is the same as that of A20.

| | name | sequence |
|---|---|---|
| Targets | W20 | 5′-/biotin/GTT GGA GCT GGT GGC GTA GG-3′ |
| | A20 | 5′-/biotin/GTT GGA GCT GCT GGC GTA GG-3′ |
| | D20 | 5′-/biotin/GTT GGA GCT GAT GGC GTA GG-3′ |
| | V20 | 5′-/biotin/GTT GGA GCT GTT GGC GTA GG-3′ |
| | A60 | 5′-/biotin/GAA TAT AAA CT T GTG GTA GTT GGA GCT GCT |
| | | GGC GTA GGC AAG AGT GCC TTG ACG ATA CAG-3′ |
| | C-W20 | 5′-/Cy5/GTT GGA GCT GGT GGC GTA GG-3′ |
| | B21 | 5′-/Cy5/GAG TTT TGG TAT TCT CTG GCG-3′ |
| | NB21 | 5′-/Cy5/GAG TTT TGG TTT TCT CTG GCG-3′ |
| Probes | W | 5′-/C12amine/CC TAC GCC ACC AGC TCC AAC-3′ |
| | A | 5′-/C12amine/CC TAC GCC AGC AGC TCC AAC-3′ |
| | D | 5′-/C12amine/CC TAC GCC ATC AGC TCC AAC-3′ |
| | V | 5′-/C12amine/CC TAC GCC AAC AGC TCC AAC-3′ |
| | AB | 5′-/C12amine/CGC CAG AGA ATA CCA AAA CTC-3′ |
| | ANB | 5′-/C12amine/CGC CAG AGA ATA CCA AAA CTC-3′ |
| Primers | Forward- | 5′-biotin-TGA CTG AAT ATA AAC TTG TGG TAG TTG GAG-3′ |
| for 80-bp | 80 bp | |
| KRAS | Reverse- | 5′-ATG ATT CTG AAT TAG CTG TAT CGT CAA GGC -3′ |
| amplicon | 80 bp | |

The genomic DNA samples, containing different allele compositions of the KRAS gene codon 12 were obtained from QIMR Berghofer Medical Research Institute (Brisbane, Australia). In order to obtain the 80-bp PCR products, a pair of forward and reverse primers (See Table 2) was used. A custom PCR protocol on a thermocycler (Cetus, Perkin Elmer) was used for DNA amplification. The thermocycling was initiated by 3 minutes of denaturation, followed by 30 thermal cycles of 95° C. for 40 s (denaturation), 55° C. for 30 s (annealing) and 72° C. for 60 s (extension), and terminated by 10 minutes of final extension at 72° C. The amplified products were purified using a nucleotide removal kit form Qiagen Inc. (Toronto, ON, Canada).

### DNA Hybridization in a CD-NBA Chip

The CD-NBA chip comprises of a PDMS slab (4 in. diameter) with 96 radial microchannels, sealed reversibly to a circular glass chip. The width of straight radially arranged channels was 200 μm and the height was 35 μm. The probe immobilization procedure was similar to the previously reported methods [19, 64, 65]. Briefly, 0.5 μL of probe solution (in 1.0M NaCl+0.15M NaHCO₃) was added to the inlet reservoirs of the CD-NBA chip, and it was placed on a rotating platform. The solutions were introduced into the radial channels by spinning the circular chip at 400 rpm for 3 min. The probe solutions were driven out from the channel after 20 min. of incubation at room temperature by spinning the chip at 1800 rpm for 1 min. Subsequently, the radial PDMS slab was peeled off, leaving behind 96 radial probe lines printed on the glass chip, which was then rinsed and dried. Thereafter, another PDMS slab with 96 spiral channels was sealed against the glass chip pre-printed with the probe lines to carry out the DNA hybridization. The target solution (1 μL), prepared in hybridization buffer (1×SSC+0.2% SDS) with a final concentration of 10 nM, was added to the inlet reservoir and then flowed in the spiral channel (100 μm wide) using a spin rate of 900 rpm. This spin rate resulted in ˜13 min. of dynamic hybridization of the targets to the complementary probes at the intersections of spiral channels with the radially arrayed probe lines. High-temperature experiments were achieved by heating the CD-NBA chip using a hot air blower. The temperature was calibrated in a separate experiment using a temperature sensor placed on the glass chip surface, sealed with the PDMS slab to it.

The washing procedure was performed after DNA hybridization. The wash solution was SSC with NaCl concentrations that range from 10 to 300 mM. The washing buffer contained either no AuNPs or AuNPs of various concentrations from 0.2 to 40 nM. In order to stabilize the AuNPs against salt aggregation, they were loaded with DNA oligonucleotides, with sequences irrelevant to the target strands, prior to addition to the wash buffer. This was performed by mixing various concentrations of 12- or 20-mer oligonucleotide with AuNPs and incubating the mix at 95° C. for 5 min. Afterwards, 2 μL of the AuNP wash buffer was added to the inlet reservoirs of the spiral channels. Dynamic wash was performed by spinning the CD-NBA chip at spinning rates of 700 to 1500 rpm. Stop-flow wash was performed by spinning the chip at 2200 rpm for 20 s in order to fill the channels with the wash buffer, incubating for 15 min. (stop-flow), and then ejecting the buffer with a spin rate at 2200 rpm for another 20 s. After washing (dynamic or stop-flow) was completed, streptavidin-Cy5 solution (50 μg/ml in 1×PBS buffer) was added to the inlet reservoir and allowed to flow in the channel by spinning at 1500 rpm. Finally, the spiral PDMS slab was peeled off from the glass chip.

The fluorescence detection was carried out by scanning the glass chip on a confocal laser fluorescent scanner (Typhoon 9410, GE Healthcare) at 10 μm resolution, as previously described [20, 22]. The excitation and emission wavelengths were 633 and 670 nm, respectively. The photomultiplier tube voltage was set at 600 V. The scanned image was analysed by IMAGEQUANT 5.2 software.

### DNA Adsorption on AuNPs

In order to study the kinetics of adsorption of DNA oligonucleotides on the surface of AuNPs, the fluorescence quenching measurement was conducted. Cy5-labeled W20 oligonucleotides (8 nM) were prepared in 1 ml of sodium citrate buffer (15 mM) in a polystyrene cuvette. The buffer contained NaCl concentrations of 0, 10, 30, 50, 70, 90, 110, 130 and 150 mM. The cuvette was placed in the holder of a spectrofluorometer (Photon Technology International). Thereafter, 1 ml of aqueous AuNP colloid (80 nM) was added to the cuvette and the content was mixed. Immediately after, the fluorescence intensity (excitation at 650 nm and emission at 670 nm) was monitored for 7 min. using the time-based mode.

### Surface Plasmon Resonance (SPR) Spectroscopy

The SPR measurements were performed on BIAcore X100 (GE Healthcare) as previously reported [21]. Briefly, the immobilisation of the amine-labelled 20-mer probes (A) was performed on the surface of a sensor chip (CMS), using a company-developed method [21, 33]. The carboxylic groups on the sensor surface were activated by an EDC/NHS mixture (1:1 v/v). Then, the amine-labeled probe molecules were immobilized on the sensor surface by running the immobilization solution containing the probe molecules (50 μM) and CTAB (0.6 mM) over the sensor surface. Finally, unreacted succinimide groups were deactivated using an ethanolamine solution (pH 8.5). The target solutions were prepared in the HBS-N buffer with DNA target concentrations of 10, 20, 40, 80 and 160 nM. The rate constants of DNA hybridization and dehybridization were determined using the multi-cycle kinetic procedure. Briefly, 10 nM target solutions were first continuously flowed for 60 s over the sensor chip surface (with immobilized probe). After hybridization, washing was achieved by a continuous flow of wash buffer over the sensor surface for 240 s. In the stringent wash experiment, the HBS-N buffer was used as the wash buffer. However, the AuNP wash buffer contain 5-nm AuNPs (10 nM) in the HBS-N buffer. The nanoparticles in the AuNP wash buffer had been previously loaded with the 20-mer oligonucleotides (stabilizers with a sequence unrelated to the target and probe), by mixing the stabilizers with AuNPs in water and then incubating the mix at 95° C. for 5 min. After each hybridization-wash cycle, the sensor surface was regenerated (all the target strands were washed away) by running an alkaline wash (50 mM NaOH) for 30 s. This cycle of hybridization, wash and regeneration was repeated for the other 4 target concentrations of 20, 40, 80, 160 nM.

Helicase-Dependent Amplification (HDA) and nanoHDA

All probe oligonucleotides, primers and gBlocks Gene Fragments (listed in Tables 2 and 3) were synthesized and modified by Integrated DNA Technologies (Coralville, Iowa). In order to obtain the 92-bp PCR products, a pair of forward and reverse primers (See Table 3) was used.

TABLE 3
the sequences of primers and probes used in HDA, nanoHDA and
DNA hybridization in the NBA chip.

| | | |
|---|---|---|
| b-DNA | Forward | 5′-/biotin/AGC CGA ATT CAA AAC ATC GTA ACT |
| (Positive | primer-b102 | GAG-3′ |
| HDA | Reverse | 5′-AAT ATT TTC CAA CAA CGCTTC TGC AAT-3′ |
| Control) | primer-b102 | |
| | Probe-b102 | 5′-/C12amine/TGG CCT CTC AAT GCT TTT TC-3′ |
| for 92-bp | Forward | 5′-/biotin/TTA TAA GGC CTG CTG AAA ATG ACT |
| KRAS | primer-92 bp | GAA-3′ |
| amplicons | | |
| | Reverse | 5′-TGA ATT AGC TGT ATC GTC AAG GCA CTC-3′ |
| | primer-92 bp | |
| | 162-bp | 5′-CAT TAT TTT TAT TAT AAG GCC TGC TGA |
| | gBlock® | AAA TGA CTG AAT ATA AAC TTG TGG TAG |
| | Gene | TTG GAG CTG GTG GCG TAG GCA AGA GTG |
| | Fragments | CCT TGA CGA TAC AGC TAA TTC AGA ATC |
| | | ATT TTG TGG ACG AAT ATG ATC CAA CAA |
| | | TAG AGG TAA ATC TTG TTT TAA TAT GCA-3′ |

All the reagents for HDA and also the b-DNA template were purchased from Biohelix Corporation (Beverly, Mass., USA). Taq DNA polymerase and PCR dNTP mix were purchased from Thermo Fisher Scientific (Waltham, Mass.). The restriction enzyme NlaIII was purchased from New England Biolabs (Ipswich, Mass.).

To setup a 25 μL of 1×HDA reaction, 2.5 μL of 10× annealing buffer, 0.75 μL of 100 mM MgSO₄, 2.5 μL of 500 mM NaCl, 1.75 μL of IsoAmp dNTP solution (200 μM dNTPs, 3 mM dATP), 1 μL of IsoAmp III enzyme mix 1 ng of DNA template, 0.75 μL of forward and reverse primer (2.5 μM), and 12.75 μL ddH₂O were pipetted into a 0.2 mL centrifuge tube. The enzyme mix consisted of 10 U of polymerase (an analog of Bst that doesn't have 3′-5′ exonuclease activity), 50 ng of helicase (Tte-UvrD), 200 ng Tte-MutL (a cofactor of helicase that stimulates and enhances the unwinding performance), and 25 ng ET-SSB, The HDA reaction mixture was briefly vortexed, followed by 30 s centrifugation at 1500 g. The HDA reaction mixture was then overlayed with 50 μL of silicone oil and incubated for 120 min (unless otherwise noted) in a water bath at 65° C. For nanoHDA experiments different amounts of AuNPs were added to the forward primer solution and the mix was kept overnight before being added to the HDA mix. The amplified products were purified using a nucleotide removal kit (Qiagen, Hilden, Germany).

### Polymerase Chain Reaction (PCR)

PCR amplification was performed on a thermocycler (Cetus, Perkin Elmer), as previously described [19]. To setup a 50 μL of 1× reaction, 5 μL of 10×PCR buffer, 3 μL of 50 mM MgCl₂, 5 μL of dNTP mix (2 mM each dNTP), 0.5 μL Taq DNA polymerase solution (1.25 U), 1 ng of DNA template, 8 μL of forward and reverse primer (2.5 μM), and 19.5 μL ddH₂O were pipetted in a 0.2 mL centrifuge tube, mixed by a brief vortex followed by 30 s centrifugation at 1500 g. The thermocycling was initiated by 3 min of denaturation, followed by 30 thermal cycles of 94° C. for 40 s (denaturation), 65° C. for 30 s (annealing) and 72° C. for 60 s (extension), and terminated by 10 min of final extension at 72° C. The amplified products were purified using a nucleotide removal kit (Qiagen).

### Capillary Gel Electrophoresis (CGE)

All the CGE experiments were performed on Bioanalyzer 2100 (Agilent Technologies, Santa Clara, Calif.). A DNA 1000 kit was used to analyze the 92-bp amplicons. Briefly, 1 μL of purified amplicons were diluted 1:10 in ddH₂O and added together with 5 μL of marker solution (low and higher markers) to the DNA chips. The electropherograms were obtained using the 2100 Expert software (Agilent Technologies). The chips may be cleaned and re-used, as previously described [66].

It is understood that the examples in the foregoing disclosure in no way serve to limit the scope of this invention, but rather are presented for illustrative purposes. As will be apparent to those skilled in the art in the light of the foregoing disclosure, many alterations and modifications are possible in the practice of this invention without departing from the scope thereof.

This invention has a wide range of aspects. Without limitation, the aspects include each of the following:

1. A nucleic acid hybridization method, comprising:
(a) immobilizing probe nucleic acid molecules on a surface;
(b) flowing target nucleic acid molecules to the immobilized probe nucleic acid molecules on said surface in a hybridization buffer solution;
(c) washing said surface with a wash solution which comprises nanoparticles; and
(d) detecting the presence of duplexes on said surface comprising a strand of one of said target nucleic acid molecules and a strand of one of said probe nucleic acid molecules.
   2. The method according to aspect 1, wherein the nanoparticles are generally spherical in shape.
   3. The method according to aspect 1 or 2, wherein the nanoparticles are sized between 1 and 10 nanometers.
   4. The method according to aspect 3, wherein the nanoparticles are sized between 3.5 to 6.5 nanometers.
   5. The method according to aspect 4, wherein the nanoparticles have an average diameter of about 5 nanometers.
   6. The method according to any one of aspects 1 to 5, wherein the nanoparticles are coated with negative charged ions.
   7. The method according to aspect 6, wherein the nanoparticles are coated with citrate.
   8. The method according to any one of aspects 1 to 7, wherein surfaces of the nanoparticles are loaded with oligonucleotide stabilizers whose sequences are irrelevant with respect to the sequences of the probe nucleic acid molecules or the target nucleic acid molecules.
   9. The method according to aspect 8, wherein the length of the oligonucleotide stabilizers is 20-mer or shorter.
   10. The method according to aspect 9, wherein the length of the oligonucleotide stabilizers is 15-mer or shorter.
   11. The method according to aspect 10, wherein the length of the oligonucleotide stabilizers is 12-mer.
   12. The method according to any one of aspects 1 to 11, wherein the concentration of the nanoparticles in the wash solution is in a range of 2 to 20 nM.
   13. The method according to any one of aspects 1 to 12, wherein the concentration of NaCl in the wash solution is in a range of 50 to 300 nM.
   14. The method according to any one of aspects 1 to 12, wherein the wash solution has an ionic strength equivalent to NaCl concentration of between 50 and 150 nM.
   15. The method according to any one of aspects 1 to 14, wherein the washing step is performed at an ambient temperature.
   16. The method according to any one of aspects 1 to 14, wherein the washing step is performed at a temperature below 30° C.
   17. The method according to aspect 16, wherein the washing step is performed at a temperature between 20° C. and 25° C.
   18. The method according to any one of aspects 1 to 17, wherein said surface is formed from a material selected from the group consisting of glass, silicon, plastic, polymer and cellulose.
   19. The method according to any one of aspects 1 to 18, wherein the probe nucleic acid molecules comprise single-stranded DNA or oligonucleotides.
   20. The method according to any one of aspects 1 to 19, wherein the target nucleic acid molecules are conjugated with a detectable label.
   21. A method for distinguishing two target nucleic acid molecules whose nucleotide sequences differ by at least one nucleotide, the method comprising:
   carrying out two separate nucleic acid hybridization assays in parallel, the first assay with a first target and a probe, the second assay with a second target and the same probe, each assay comprising:
(a) providing a sample solution comprising a target nucleic acid;
(b) incubating said sample solution with probe nucleic acid molecules immobilized on a surface;
(c) washing said surface with a wash solution which comprises nanoparticles; and
(d) detecting the presence of target:probe duplex on the surface;
   whereby the two target nucleic acid molecules are distinguished by different degrees of hybridization to the probe.
   22. A microarray method comprising:
(a) providing a solid support;
(b) immobilizing a plurality of nucleic acid probes at discrete positions on the support;
(c) exposing a sample solution to the probes, the sample solution comprising sample nucleic acid molecules;
(d) washing off the sample solution with a wash solution which comprises nanoparticles; and
(e) determining the degree of hybridization between the sample molecules and the probes.
   23. A method of using a microfluidic microarray assembly (MMA) comprising:
   (a) providing a test chip;
   (b) providing a first channel plate sealingly connectable to said test chip for applying at least one probe reagent to said test chip, wherein said first channel plate comprises a plurality of first microfluidic channels configured in a first predetermined reagent pattern;
   (c) assembling said first channel plate to said test chip;
   (d) flowing said at least one probe reagent through said first microfluidic channels to form a first array of said at least one probe reagent on said test chip in said first predetermined reagent pattern;
   (e) immobilizing said at least one probe reagent on said test chip;
   (f) removing said first channel plate from said test chip;
   (g) providing a second channel plate sealingly connectable to said test chip for applying at least one sample reagent to said test chip, wherein said second channel plate comprises a plurality of second microfluidic channels configured in a second predetermined pattern differing from said first predetermined pattern;
   (h) assembling said second channel plate to said test chip;
   (i) flowing said at least one sample reagent through said second microfluidic channels to form a second array, wherein said second array intersects said first array at said test locations;
   (j) flowing a wash solution which comprises nanoparticles through said second microfluidic channels; and
   (k) detecting any hybridization products at said test locations.
   24. The method according to aspect 23, wherein said at least one probe reagent comprises a plurality of different probes, wherein each of said probes is flowable through separate ones of said first microfluidic channels.
   25. The method according to aspect 23 or 24, wherein said at least one sample reagent comprises a plurality of different test samples, wherein each of said samples is flowable through separate ones of said second microfluidic channels.
   26. The method according to any one of aspects 23 to 25, wherein one of said first and second predetermined reagent patterns is a radial pattern and the other of said first and second predetermined reagent patterns is a spiral pattern.
   27. The method according to any one of aspects 23 to 26, wherein the nanoparticles are generally spherical in shape.
   28. The method according to any one of aspects 23 to 27, wherein the nanoparticles are sized between 1 and 10 nanometers.
   29. The method according to aspect 28, wherein the nanoparticles are sized between 3.5 to 6.5 nanometers.
   30. The method according to aspect 29, wherein the nanoparticles have an average diameter of about 5 nanometers.
   31. The method according to any one of aspects 23 to 30, wherein the nanoparticles are coated with negative charged ions.
   32. The method according to aspect 31, wherein the nanoparticles are coated with citrate.
   33. The method according to any one of aspects 23 to 32, wherein surfaces of the nanoparticles are loaded with oligonucleotide stabilizers whose sequences are irrelevant with respect to the sequences of the probes or the samples.
   34. The method according to aspect 33, wherein the length of the oligonucleotide stabilizers is 20-mer or shorter.
   35. The method according to aspect 34, wherein the length of the oligonucleotide stabilizers is 15-mer or shorter.
   36. The method according to aspect 35, wherein the length of the oligonucleotide stabilizers is 12-mer.
   37. The method according to any one of aspects 23 to 36, wherein the concentration of the nanoparticles in the wash solution is in a range of 2 to 20 nM.
   38. The method according to any one of aspects 23 to 37, wherein the concentration of NaCl in the wash solution is in a range of 50 to 300 nM.
   39. The method according to any one of aspects 23 to 37, wherein the wash solution has an ionic strength equivalent to NaCl concentration of between 50 and 150 nM.
   40. The method according to any one of aspects 23 to 39, wherein the washing step is performed at an ambient temperature.
   41. The method according to any one of aspects 23 to 39, wherein the washing step is performed at a temperature below 30° C.
   42. The method according to aspect 41, wherein the washing step is performed at a temperature between 20° C. and 25° C.
   43. The method according to any one of aspects 23 to 42, wherein said test chip is formed from a material selected from the group consisting of glass, silicon, plastic, polymer and cellulose.
   44. The method according to any one of aspects 23 to 43, wherein the probes comprise single-stranded DNA or oligonucleotides.
   45. The method according to any one of aspects 23 to 44, wherein the samples are conjugated with a detectable label.
   46. The method according to any one of aspects 1 to 45, wherein the nanoparticles comprise metal nanoparticles.
   47. The method according to any one of aspects 1 to 45, wherein the nanoparticles comprise non-metal nanoparticles.
   48. The method according to any one of aspects 1 to 45, wherein the nanoparticles comprise gold nanoparticles.
   49. The method according to any one of aspects 1 to 45, wherein the nanoparticles comprise silver nanoparticles.
   50. An isothermal nucleic acid amplification method, the method comprising:
   (i) providing substrate nucleic acid molecules in a reaction solution which comprises a helicase, a polymerase, dNTPs, oligonucleotide primers, and nanoparticles,
   (ii) allowing the substrate nucleic acid molecules to be denatured by the helicase,
   (iii) allowing the oligonucleotide primers to anneal to the denatured substrate nucleic acid molecules,
   (iv) allowing the polymerase to extend the annealed primers to synthesize complementary nucleic acid strands to form duplex products, and
   (v) repeating steps (a) through (d) for a plurality of cycles to amplify the substrate nucleic acid molecules,
   wherein the presence of the nanoparticles in the reaction solution enhances the denaturation of the substrate nucleic acid molecules and increases the amount of the amplified products.
   51. The method according to aspect 50, further comprising digesting the substrate nucleic acid molecules with a restriction enzyme prior to step (i).
   52. The method according to aspect 5, wherein the substrate nucleic acid molecules are digested with a restriction enzyme to fragment sizes of less than 500 bp.
   53. The method according to aspect 6, wherein the substrate nucleic acid molecules are digested with a restriction enzyme to fragment sizes of less than 300 bp.
   54. The method according to any one of aspects 50 to 53, wherein the concentration of the nanoparticles in the reaction solution is in a range of 0.1 to 10 nM.
   55. The method according to any one of aspects 50 to 54, wherein the nanoparticles in the reaction solution have an average diameter of about 5 to 10 nanometers.
   56. The method according to any one of aspects 50 to 55, wherein the steps (i) through (v) are carried out at a constant reaction temperature.
   57. The method according to aspect 56, wherein the constant reaction temperature is in a range of 40 to 70° C.
   58. The method according to aspect 57, wherein the constant reaction temperature is about 65° C.
   59. The method according to any one of aspects 50 to 58, wherein the oligonucleotide primers are a pair of oligonucleotide primers wherein one primer hybridizes to a first end and one primer hybridizes to a second end of the substrate nucleic acid to be amplified.
   60. The method according to any one of aspects 50 to 59, wherein the helicase comprises a plurality of helicases.
   61. The method according to aspect 60, wherein the helicases comprise a 3′ to 5′ helicase, a 5′ to 3′ helicase, or both.
   62. The method according to any one of aspects 50 to 61, wherein the reaction solution comprises a single strand binding (SSB) protein.
   63. The method according to any one of aspects 50 to 62, wherein the polymerase is a Klenow fragment of E. coli DNA polymerase I, T7 DNA polymerase, Bst polymerase large fragment, or a homolog thereof.
   64. The method according to any one of aspects 1 to 20, wherein the target nucleic acid molecules are amplified using an isothermal nucleic acid amplification method, the amplification method comprising:
   (i) providing substrate nucleic acid molecules in a reaction solution which comprises a helicase, a polymerase, dNTPs, oligonucleotide primers, and nanoparticles,
   (ii) allowing the substrate nucleic acid molecules to be denatured by the helicase,
   (iii) allowing the oligonucleotide primers to anneal to the denatured substrate nucleic acid molecules,
   (iv) allowing the polymerase to extend the annealed primers to synthesize complementary nucleic acid strands to form duplex products, and
   (v) repeating steps (i) through (iv) for a plurality of cycles to amplify the substrate nucleic acid molecules,
   wherein the presence of the nanoparticles in the reaction solution enhances the denaturation of the substrate nucleic acid molecules and increases the amount of the amplified products, and
   wherein the concentration and other parameters of the nanoparticles in the amplification method and the washing step in the hybridization method are independently optimized.
   65. The method according to aspect 22, wherein the sample nucleic acid molecules are amplified using an isothermal nucleic acid amplification method, the amplification method comprising:
   (i) providing substrate nucleic acid molecules in a reaction solution which comprises a helicase, a polymerase, dNTPs, oligonucleotide primers, and nanoparticles,
   (ii) allowing the substrate nucleic acid molecules to be denatured by the helicase,
   (iii) allowing the oligonucleotide primers to anneal to the denatured substrate nucleic acid molecules,
   (iv) allowing the polymerase to extend the annealed primers to synthesize complementary nucleic acid strands to form duplex products, and
   (v) repeating steps (i) through (iv) for a plurality of cycles to amplify the substrate nucleic acid molecules,
   wherein the presence of the nanoparticles in the reaction solution enhances the denaturation of the substrate nucleic acid molecules and increases the amount of the amplified products, and
   wherein the concentration and other parameters of the nanoparticles in the amplification method and the washing step in the hybridization method are independently optimized.
   66. The method according to aspect 23, wherein said at least one sample reagent comprises nucleic acid molecules which are amplified using an isothermal nucleic acid amplification method, the amplification method comprising:
   (i) providing substrate nucleic acid molecules in a reaction solution which comprises a helicase, a polymerase, dNTPs, oligonucleotide primers, and nanoparticles,
   (ii) allowing the substrate nucleic acid molecules to be denatured by the helicase,
   (iii) allowing the oligonucleotide primers to anneal to the denatured substrate nucleic acid molecules,
   (iv) allowing the polymerase to extend the annealed primers to synthesize complementary nucleic acid strands to form duplex products, and
   (v) repeating steps (i) through (iv) for a plurality of cycles to amplify the substrate nucleic acid molecules,
   wherein the presence of the nanoparticles in the reaction solution enhances the denaturation of the substrate nucleic acid molecules and increases the amount of the amplified products, and
   wherein the concentration and other parameters of the nanoparticles in the amplification method and the washing step in the hybridization method are independently optimized.

### REFERENCES

1. V. Gubala, L. F. Harris, A. J. Ricco, M. X. Tan and D. E. Williams, Point of care diagnostics: status and future. Analytical chemistry, 2011, 84, 487-515.
2. A. Niemz, T. M. Ferguson and D. S. Boyle, Point-of-care nucleic acid testing for infectious diseases. Trends in biotechnology, 2011, 29, 240-250.
3. K. Knez, D. Spasic, K. P. Janssen and J. Lammertyn, Emerging technologies for hybridization based single nucleotide polymorphism detection. Analyst, 2014, 139, 353-370.
4. H. Koltai and C. Weingarten-Baror, Specificity of DNA microarray hybridization: characterization, effectors and approaches for data correction. Nucleic acids research, 2008, 36, 2395-2405.
5. A. Chagovetz and S. Blair, Real-time DNA microarrays: reality check. Biochemical Society Transactions, 2009, 37, 471-475.
6. C. T. Wittwer, High-resolution DNA melting analysis: advancements and limitations. Human mutation, 2009, 30, 857-859.
7. H. Urakawa, S. El Fantroussi, H. Smidt, J. C. Smoot, E. H. Tribou, J. J. Kelly, P. A. Noble and D. A. Stahl, Optimization of single-base-pair mismatch discrimination in oligonucleotide microarrays. Applied and environmental microbiology, 2003, 69, 2848-2856.
8. L. A. Marcelino, V. Backman, A. Donaldson, C. Steadman, J. R. Thompson, S. P. Preheim, C. Lien, E. Lim, D. Veneziano and M. F. Polz, Accurately quantifying low-abundant targets amid similar sequences by revealing hidden correlations in oligonucleotide microarray data. Proceedings of the National Academy of Sciences, 2006, 103, 13629-13634.
9. R. A. Rule, A. E. Pozhitkov and P. A. Noble, Use of hidden correlations in short oligonucleotide array data are insufficient for accurate quantification of nucleic acid targets in complex target mixtures. Journal of microbiological methods, 2009, 76, 188-195.
10. S. Michiels, S. Koscielny and C. Hill, Prediction of cancer outcome with microarrays: a multiple random validation strategy. The Lancet, 2005, 365, 488-492.
11. V. V. Demidov and M. D. Frank-Kamenetskii, Two sides of the coin: affinity and specificity of nucleic acid interactions. Trends in biochemical sciences, 2004, 29, 62-71.
12. L. Poulsen, M. J. Søe, D. Snakenborg, L. B. Møller and M. Dufva, Multi-stringency wash of partially hybridized 60-mer probes reveals that the stringency along the probe decreases with distance from the microarray surface. Nucleic acids research, 2008, 36, e132-e132.
13. J. Grimes, Y. V. Gerasimova and D. M. Kolpashchikov, Real-time SNP Analysis in Secondary-Structure-Folded Nucleic Acids. Angewandte Chemie, 2010, 122, 9134-9137.
14. D. M. Kolpashchikov, Binary malachite green aptamer for fluorescent detection of nucleic acids. Journal of the American Chemical Society, 2005, 127, 12442-12443.
15. D. M. Kolpashchikov, A binary DNA probe for highly specific nucleic acid recognition. Journal of the American Chemical Society, 2006, 128, 10625-10628.
16. S. A. Marras, F. R. Kramer and S. Tyagi, Efficiencies of fluorescence resonance energy transfer and contact-mediated quenching in oligonucleotide probes. Nucleic acids research, 2002, 30, e122-e122.
17. S. Tyagi and F. R. Kramer, Molecular beacons: probes that fluoresce upon hybridization. Nature biotechnology, 1996, 14, 303-308.
18. D. Y. Zhang, S. X. Chen and P. Yin, Optimizing the specificity of nucleic acid hybridization. Nature chemistry, 2012, 4, 208-214.
19. A. Sedighi and P. C. Li, KRAS gene codon 12 mutation detection enabled by gold nanoparticles conducted in a nanobioarray chip. Analytical biochemistry, 2014, 448, 58-64.
20. L. Wang and P. C. Li, Gold nanoparticle-assisted single base-pair mismatch discrimination on a microfluidic microarray device. Biomicrofluidics, 2010, 4, 032209, 1-9.
21. A. Sedighi, P. C. Li, I. C. Pekcevik and B. D. Gates, A Proposed Mechanism of the Influence of Gold Nanoparticles on DNA Hybridization. ACS Nano, 2014, 8, 6765-6777.
22. L. Wang and P. C. Li, Optimization of a microfluidic microarray device for the fast discrimination of fungal pathogenic DNA. Analytical biochemistry, 2010, 400, 282-288.
23. L. Wang, P. C. Li, H.-Z. Yu and A. M. Parameswaran, Fungal pathogenic nucleic acid detection achieved with a microfluidic microarray device. Analytica chimica acta, 2008, 610, 97-104.
24. J. Lee Rodgers and W. A. Nicewander, Thirteen ways to look at the correlation coefficient. The American Statistician, 1988, 42, 59-66.
25. A. Lomakin and M. D. Frank-Kamenetskii, A theoretical analysis of specificity of nucleic acid interactions with oligonucleotides and peptide nucleic acids (PNAs). Journal of molecular biology, 1998, 276, 57-70.
26. D. R. Kearns and T. L. James, NMR Studies of Conformational States and Dynamics of DN. Critical Reviews in Biochemistry and Molecular Biology, 1984, 15, 237-290.
27. T. Dauxois, M. Peyrard and A. Bishop, Entropy-driven DNA denaturation. Physical Review E, 1993, 47, R44-R47.
28. Y. Zeng and G. Zocchi, Mismatches and bubbles in DNA. Biophysical journal, 2006, 90, 4522-4529.
29. X. Zhang, M. R. Servos and J. Liu, Surface science of DNA adsorption onto citrate-capped gold nanoparticles. Langmuir, 2012, 28, 3896-3902.
30. L. M. Demers, M. Östblom, H. Zhang, N.-H. Jang, B. Liedberg and C. A. Mirkin, Thermal desorption behavior and binding properties of DNA bases and nucleosides on gold. Journal of the American Chemical Society, 2002, 124, 11248-11249.
31. B. L. Parsons and M. B. Myers, Personalized cancer treatment and the myth of KRAS wild-type colon tumors. Discovery medicine, 2013, 15, 259-267.
32. C. Koch, P. C. Li and R. Utkhede, Evaluation of thin films of agarose on glass for hybridization of DNA to identify plant pathogens with microarray technology. Analytical biochemistry, 2005, 342, 93-102.
33. S. Löfås and A. Mcwhirter, The art of immobilization for SPR sensors. in Surface Plasmon Resonance Based Sensors, Springer, 2006, pp. 117-151.
34. Saiki, R. K.; Scharf, S.; Faloona, F.; Mullis, K. B.; Horn, G. T.; Erlich, H. A.; Arnheim, N., Enzymatic amplification of beta-globin genomic sequences and restriction site analysis for diagnosis of sickle cell anemia. Science 1985, 230 (4732), 1350-1354.
35. Notomi, T.; Okayama, H.; Masubuchi, H.; Yonekawa, T.; Watanabe, K.; Amino, N.; Hase, T., Loop-mediated isothermal amplification of DNA. Nucleic acids research 2000, 28 (12), e63, 1-7.
36. Lizardi, P. M.; Huang, X.; Zhu, Z.; Bray-Ward, P.; Thomas, D. C.; Ward, D. C., Mutation detection and single-molecule counting using isothermal rolling-circle amplification. Nature genetics 1998, 19 (3), 225-232.
37. Kievits, T.; van Gemen, B.; van Strijp, D.; Schukkink, R.; Dircks, M.; Adriaanse, H.; Malek, L.; Sooknanan, R.; Lens, P., NASBA TM isothermal enzymatic in vitro nucleic acid amplification optimized for the diagnosis of HIV-1 infection. Journal of virological methods 1991, 35 (3), 273-286.
38. Walker, G. T.; Fraiser, M. S.; Schram, J. L.; Little, M. C.; Nadeau, J. G.; Malinowski, D. P., Strand displacement amplification—an isothermal, in vitro DNA amplification technique. Nucleic acids research 1992, 20 (7), 1691-1696.
39. Vincent, M.; Xu, Y.; Kong, H., Helicase-dependent isothermal DNA amplification. EMBO reports 2004, 5(8), 795-800.
40. Hicke, B.; Pasko, C.; Groves, B.; Ager, E.; Corpuz, M.; Frech, G.; Munns, D.; Smith, W.; Warcup, A.; Denys, G., Automated detection of toxigenic Clostridium difficile in clinical samples: isothermal tcdB amplification coupled to array-based detection. Journal of clinical microbiology 2012, 50 (8), 2681-2687.
41. Andresen, D.; von Nickisch-Rosenegk, M.; Bier, F. F., Helicase dependent OnChip-amplification and its use in multiplex pathogen detection. Clinica Chimica Acta 2009, 403 (1), 244-248.
42. Chow, W. H. A.; McCloskey, C.; Tong, Y.; Hu, L.; You, Q.; Kelly, C. P.; Kong, H.; Tang, Y.-W.; Tang, W., Application of isothermal helicase-dependent amplification with a disposable detection device in a simple sensitive stool test for toxigenic Clostridium difficile. The Journal of Molecular Diagnostics 2008, 10 (5), 452-458.
43. Gill, P.; Alvandi, A.-H.; Abdul-Tehrani, H.; Sadeghizadeh, M., Colorimetric detection of Helicobacter pylori DNA using isothermal helicase-dependent amplification and gold nanoparticle probes. Diagnostic microbiology and infectious disease 2008, 62 (2), 119-124.
44. Goldmeyer, J.; Li, H.; McCormac, M.; Cook, S.; Stratton, C.; Lemieux, B.; Kong, H.; Tang, W.; Tang, Y.-W., Identification of Staphylococcus aureus and determination of methicillin resistance directly from positive blood cultures by isothermal amplification and a disposable detection device. Journal of clinical microbiology 2008, 46 (4), 1534-1536.
45. Kivlehan, F.; Mavré, F.; Talini, L.; Limoges, B.; Marchal, D., Real-time electrochemical monitoring of isothermal helicase-dependent amplification of nucleic acids. Analyst 2011, 136 (18), 3635-3642.
46. Mahalanabis, M.; Do, J.; ALMuayad, H.; Zhang, J. Y.; Klapperich, C. M., An integrated disposable device for DNA extraction and helicase dependent amplification. Biomedical microdevices 2010, 12 (2), 353-359.
47. Motré, A.; Li, Y.; Kong, H., Enhancing helicase-dependent amplification by fusing the helicase with the DNA polymerase. Gene 2008, 420 (1), 17-22.
48. Torres-Chavolla, E.; Alocilja, E. C., Nanoparticle based DNA biosensor for tuberculosis detection using thermophilic helicase-dependent isothermal amplification. Biosensors and Bioelectronics 2011, 26 (11), 4614-4618.
49. Ramalingam, N.; San, T. C.; Kai, T. J.; Mak, M. Y. M.; Gong, H.-Q., Microfluidic devices harboring unsealed reactors for real-time isothermal helicase-dependent amplification. Microfluidics and nanofluidics 2009, 7 (3), 325-336.
50. Li, Y.; Jortani, S. A.; Ramey-Hartung, B.; Hudson, E.; Lemieux, B.; Kong, H., Genotyping three SNPs affecting warfarin drug response by isothermal real-time HDA assays. Clinica Chimica Acta 2011, 412 (1), 79-85.
51. Yuce, M.; Kurt, H.; Mokkapati, V. R.; Budak, H., Employment of nanomaterials in polymerase chain reaction: insight into the impacts and putative operating mechanisms of nano-additives in PCR. RSC Advances 2014, 4 (69), 36800-36814.
52. Lou, X.; Zhang, Y., Mechanism studies on nanoPCR and applications of gold nanoparticles in genetic analysis. ACS applied materials & interfaces 2013, 5 (13), 6276-6284.
53. Li, H.; Huang, J.; Lv, J.; An, H.; Zhang, X.; Zhang, Z.; Fan, C.; Hu, J., nanoparticle PCR: Nanogold-Assisted PCR with Enhanced Specifity. Angewandte Chemie International edition 2005, 44(32), 5100-5103.
54. Li, M.; Lin, Y.-C.; Wu, C.-C.; Liu, H.-S., Enhancing the efficiency of a PCR using gold nanoparticles. Nucleic acids research 2005, 33 (21), e184, 1-10.
55. Vu, B. V.; Litvinov, D.; Willson, R. C., Gold nanoparticle effects in polymerase chain reaction: favoring of smaller products by polymerase adsorption. Analytical chemistry 2008, 80 (14), 5462-5467.
56. Mi, L.; Wen, Y.; Pan, D.; Wang, Y.; Fan, C.; Hu, J., Modulation of DNA Polymerase with Gold Nanoparticles and their Applications in Hot-Start PCR. Small 2009, 5(22), 2597-2600.
57. Tong, Y.; Lemieux, B.; Kong, H., Multiple strategies to improve sensitivity, speed and robustness of isothermal nucleic acid amplification for rapid pathogen detection. BMC biotechnology 2011, 11 (1), 50, 1-7.
58. Chen, C.; Wang, W.; Ge, J.; Zhao, X. S., Kinetics and thermodynamics of DNA hybridization on gold nanoparticles. Nucleic acids research 2009, 37 (11), 3756-3765.
59. Cho, K.; Lee, Y.; Lee, C.-H.; Lee, K.; Kim, Y.; Choi, H.; Ryu, P.-D.; Lee, S. Y.; Joo, S.-W., Selective aggregation mechanism of unmodified gold nanoparticles in detection of single nucleotide polymorphism. The Journal of Physical Chemistry C 2008, 112 (23), 8629-8633.
60. Wan, W.; Yeow, J. T., The effects of gold nanoparticles with different sizes on polymerase chain reaction efficiency. Nanotechnology 2009, 20 (32), 325702, 1-5.
61. Mandal, S.; Hossain, M.; Muruganandan, T.; Kumar, G. S.; Chaudhuri, K., Gold nanoparticles alter Taq DNA polymerase activity during polymerase chain reaction. RSC Advances 2013, 3 (43), 20793-20799.
62. An, L.; Tang, W.; Ranalli, T. A.; Kim, H.-J.; Wytiaz, J.; Kong, H., Characterization of a thermostable UvrD helicase and its participation in helicase-dependent amplification. Journal of Biological Chemistry 2005, 280 (32), 28952-28958.
63. Zhao, W.; Lee, T. M.; Leung, S. S.; Hsing, I.-M., Tunable stabilization of gold nanoparticles in aqueous solutions by mononucleotides. Langmuir 2007, 23 (13), 7143-7147.
64. Sedighi, A.; Li, P. C., Gold nanoparticle assists SNP detection at room temperature in the nanoBioArray chip. International Journal of Materials Science and Engineering 2013, 1 (1), 45-49.
65. Wang, L.; Li, P. C., Flexible microarray construction and fast DNA hybridization conducted on a microfluidic chip for greenhouse plant fungal pathogen detection. Journal of agricultural and food chemistry 2007, 55 (26), 10509-10516.
66. Chim, W.; Li, P. C., Repeated capillary electrophoresis separations conducted on a commercial DNA chip. Analytical Methods 2012, 4 (3), 864-868.
   All references cited herein are hereby incorporated by reference. Additionally, U.S. Pat. No. 8,343,778 entitled “microfluidic microarray assemblies and methods of manufacturing and using” and US Patent Application Publication No. US 2012/0108451 entitled “methods and apparatus for nanoparticle-assisted nucleic acid hybridization and microarray analysis” are hereby incorporated by reference.

What is claimed is:

1. A nucleic acid hybridization method, comprising:
a) immobilizing probe nucleic acid molecules on a surface;
b) flowing target nucleic acid molecules to the immobilized probe nucleic acid molecules on said surface in a hybridization buffer solution;
c) washing said surface with a wash solution which comprises nanoparticles; and
d) detecting the presence of duplexes on said surface comprising a strand of one of said target nucleic acid molecules and a strand of one of said probe nucleic acid molecules.

2. The method according to claim 1, wherein prior to the hybridization method the target nucleic acid molecules are generated using an isothermal nucleic acid amplification method, the amplification method comprising:
(i) providing substrate nucleic acid molecules in a reaction solution which comprises a helicase, a polymerase, dNTPs, oligonucleotide primers, and nanoparticles,
(ii) allowing the substrate nucleic acid molecules to be denatured by the helicase,
(iii) allowing the oligonucleotide primers to anneal to the denatured substrate nucleic acid molecules,
(iv) allowing the polymerase to extend the annealed primers to synthesize complementary nucleic acid strands to generate duplex molecules, and
(v) repeating steps (i) through (iv) for a plurality of cycles to amplify the substrate nucleic acid molecules,
wherein the presence of the nanoparticles in the reaction solution enhances the denaturation of the substrate nucleic acid molecules and increases the amount of the amplified products, and
wherein the concentration and other parameters of the nanoparticles in the amplification method and the washing step of the hybridization method are independently optimized.

3. The method according to claim 1, wherein the nanoparticles are generally spherical in shape.

4. The method according to claim 3, wherein the nanoparticles are sized between 1 and 10 nanometers.

5. The method according to claim 4, wherein the nanoparticles have an average diameter of about 5 nanometers.

6. The method according to claim 1, wherein the nanoparticles are coated with negative charged ions.

7. The method according to claim 6, wherein the nanoparticles are coated with citrate.

8. The method according to claim 1, wherein surfaces of the nanoparticles are loaded with oligonucleotide stabilizers whose sequences are irrelevant with respect to the sequences of the probe nucleic acid molecules or the target nucleic acid molecules.

9. The method according to claim 8, wherein the length of the oligonucleotide stabilizers is 20-mer or shorter.

10. The method according to claim 9, wherein the length of the oligonucleotide stabilizers is 15-mer or shorter.

11. The method according to claim 10, wherein the length of the oligonucleotide stabilizers is 12-mer.

12. The method according to claim 1, wherein the concentration of the nanoparticles in the wash solution is in a range of 2 to 20 nM.

13. The method according to claim 1, wherein the concentration of NaCl in the wash solution is in a range of 50 to 300 nM.

14. The method according to claim 1, wherein the nanoparticles comprise gold nanoparticles.

15. The method according to claim 1, wherein the washing step is performed at an ambient temperature.

16. The method according to claim 1, wherein the washing step is performed at a temperature below 30° C.

17. The method according to claim 16, wherein the washing step is performed at a temperature between 20° C. and 25° C.

18. A microarray method comprising:
a) providing a solid support;
b) immobilizing a plurality of nucleic acid probes at discrete positions on the support;
c) exposing a sample solution to the probes, the sample solution comprising sample nucleic acid molecules;
d) washing off the sample solution with a wash solution which comprises nanoparticles; and
e) determining the degree of hybridization between the sample molecules and the probes.

19. The method according to claim 18, wherein prior to the microarray method the sample nucleic acid molecules are generated using an isothermal nucleic acid amplification method, the amplification method comprising:
(i) providing substrate nucleic acid molecules in a reaction solution which comprises a helicase, a polymerase, dNTPs, oligonucleotide primers, and nanoparticles,
(ii) allowing the substrate nucleic acid molecules to be denatured by the helicase,
(iii) allowing the oligonucleotide primers to anneal to the denatured substrate nucleic acid molecules,
(iv) allowing the polymerase to extend the annealed primers to synthesize complementary nucleic acid strands to generate duplex molecules, and
(v) repeating steps (i) through (iv) for a plurality of cycles to amplify the substrate nucleic acid molecules,
wherein the presence of the nanoparticles in the reaction solution enhances the denaturation of the substrate nucleic acid molecules and increases the amount of the amplified products, and
wherein the concentration and other parameters of the nanoparticles in the amplification method and the washing step in the microarray method are independently optimized.

20. A method of using a microfluidic microarray assembly (MMA) comprising:
(a) providing a test chip;
(b) providing a first channel plate sealingly connectable to said test chip for applying at least one probe reagent to said test chip, wherein said first channel plate comprises a plurality of first microfluidic channels configured in a first predetermined reagent pattern;
(c) assembling said first channel plate to said test chip;
(d) flowing said at least one probe reagent through said first microfluidic channels to form a first array of said at least one probe reagent on said test chip in said first predetermined reagent pattern;
(e) immobilizing said at least one probe reagent on said test chip;
(f) removing said first channel plate from said test chip;
(g) providing a second channel plate sealingly connectable to said test chip for applying at least one sample reagent to said test chip, wherein said second channel plate comprises a plurality of second microfluidic channels configured in a second predetermined pattern differing from said first predetermined pattern;
(h) assembling said second channel plate to said test chip;
(i) flowing said at least one sample reagent through said second microfluidic channels to form a second array, wherein said second array intersects said first array at said test locations;
(j) flowing a wash solution which comprises nanoparticles through said second microfluidic channels; and
(k) detecting any hybridization products at said test locations.

21. The method according to claim 20, wherein said at least one probe reagent comprises a plurality of different probes, wherein each of said probes is flowable through separate ones of said first microfluidic channels.

22. The method according to claim 21, wherein said at least one sample reagent comprises a plurality of different test samples, wherein each of said samples is flowable through separate ones of said second microfluidic channels.

23. The method according to claim 20, wherein one of said first and second predetermined reagent patterns is a radial pattern and the other of said first and second predetermined reagent patterns is a spiral pattern.

24. The method according to claim 20, wherein said at least one sample reagent comprises nucleic acid molecules which are generated using an isothermal nucleic acid amplification method prior to the MMA method, the amplification method comprising:
(i) providing substrate nucleic acid molecules in a reaction solution which comprises a helicase, a polymerase, dNTPs, oligonucleotide primers, and nanoparticles,
(ii) allowing the substrate nucleic acid molecules to be denatured by the helicase,
(iii) allowing the oligonucleotide primers to anneal to the denatured substrate nucleic acid molecules,
(iv) allowing the polymerase to extend the annealed primers to synthesize complementary nucleic acid strands to generate duplex molecules, and
(v) repeating steps (i) through (iv) for a plurality of cycles to amplify the substrate nucleic acid molecules,
wherein the presence of the nanoparticles in the reaction solution enhances the denaturation of the substrate nucleic acid molecules and increases the amount of the amplified products, and
wherein the concentration and other parameters of the nanoparticles in the amplification method and the washing step in the MMA method are independently optimized.


SEQUENCE LISTING (21 sequences)

SEQ ID NO: 1 - DNA, 20 bp, Homo sapiens
gttggagctggtggcgtagg

SEQ ID NO: 2 - DNA, 20 bp, Homo sapiens
gttggagctgctggcgtagg

SEQ ID NO: 3 - DNA, 20 bp, Homo sapiens
gttggagctgatggcgtagg

SEQ ID NO: 4 - DNA, 20 bp, Homo sapiens
gttggagctgttggcgtagg

SEQ ID NO: 5 - DNA, 60 bp, Homo sapiens
gaatataaacttgtggtagttggagctgctggcgtaggcaagagtgccttgacgatacag

SEQ ID NO: 6 - DNA, 21 bp, botrytis cinerea
gagttttggtattctctggcg

SEQ ID NO: 7 - DNA, 21 bp, botrytis cinerea
gagttttggttttctctggcg

SEQ ID NO: 8 - DNA, 20 bp, Homo sapiens
cctacgccaccagctccaac

SEQ ID NO: 9 - DNA, 20 bp, Homo sapiens
cctacgccagcagctccaac

SEQ ID NO: 10 - DNA, 20 bp, Homo sapiens
cctacgccatcagctccaac

SEQ ID NO: 11 - DNA, 20 bp, Homo sapiens
cctacgccaacagctccaac

SEQ ID NO: 12 - DNA, 21 bp, botrytis cinerea
cgccagagaataccaaaactc

SEQ ID NO: 13 - DNA, 21 bp, botrytis cinerea
cgccagagaataccaaaactc

SEQ ID NO: 14 - DNA, 30 bp, Homo sapiens
tgactgaatataaacttgtggtagttggag

SEQ ID NO: 15 - DNA, 30 bp, Homo sapiens
atgattctgaattagctgtatcgtcaaggc

SEQ ID NO: 16 - DNA, 27 bp, Neisseria gonorrhoeae
agccgaattcaaaacatcgtaactgag

SEQ ID NO: 17 - DNA, 27 bp, Neisseria gonorrhoeae
aatattttccaacaacgcttctgcaat

SEQ ID NO: 18 - DNA, 20 bp, Neisseria gonorrhoeae
tggcctctcaatgctttttc

SEQ ID NO: 19 - DNA, 27 bp, Homo sapiens
ttataaggcctgctgaaaatgactgaa

SEQ ID NO: 20 - DNA, 27 bp, Homo sapiens
tgaattagctgtatcgtcaaggcactc

SEQ ID NO: 21 - DNA, 162 bp, Homo sapiens
cattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgca